(19) United States
(12) Patent Application Publication
(10) Pub. No.: US 2009/0299378 A1
(43) Pub. Date: Dec. 3, 2009

(54) CONTROLLED DEPLOYMENT HANDLES FOR BONE STABILIZATION DEVICES

(76) Inventor: Peter G. Knopp, Pleasanton, CA (US)

Correspondence Address: SHAY GLENN LLP, 2755 CAMPUS DRIVE, SUITE 210, SAN MATEO, CA, 94403, US

(21) Appl. No.: 12/476,924

(22) Filed: Jun. 2, 2009

Related U.S. Application Data
(60) Provisional application No. 61/058,157, filed on Jun. 2, 2008.
(60) Provisional application No. 61/142,552, filed on Jan. 5, 2009.

(51) Int. Cl. A61F 11/00; B25B 15/04

(52) U.S. Cl. 606/108; 81/58

(57) ABSTRACT

Described herein are applicators for the delivery and/or retrieval of a bone stabilization device, as well as systems or kits including such applicators. In general, these applicators include a proximal handle and an elongate cannula configured as a linkage member connecting to the implant. The handles described herein typically include a control for regulating/controlling the release of the stabilization device. Stabilization devices are typically self-expanding devices, and the control may regulate the self-expansion so that the rate and degree of self-expansion allowed is regulated. The handles may be lockable, and may include a latch or other locking structure. These handles may also include ratcheting mechanism or other controlled expansion/release mechanism. In some variations the devices include a failsafe release configured to release either the applicator and/or the device.

### CROSS REFERENCE TO RELATED APPLICATIONS

This application claims priority to U.S. Provisional Patent Application Ser. No. 61/058,157, filed on Jun. 2, 2008, entitled “CONTROLLED DEPLOYMENT HANDLE FOR BONE STABILIZATION DEVICES”, and U.S. Provisional Patent Application Ser. No. 61/142,552, filed on Jan. 5, 2009, entitled “CONTROLLED DEPLOYMENT HANDLE FOR BONE STABILIZATION DEVICES.”

This application is related to U.S. patent application Ser. No. 11/468,759, filed on Aug. 30,2006, entitled “IMPLANTABLE DEVICES AND METHODS FOR TREATING MICRO-ARCHITECTURE DETERIORATION OF BONE TISSUE”, which claims the benefit of U.S. Provisional Application Ser. No. 60/713,259, filed on Aug. 31, 2005, entitled “IMPLANTABLE DEVICE FOR TREATING VCF, TOOLS AND METHODS”. This application is also related to U.S. patent application Ser. No. 12/041,607 filed on Mar. 3, 2008, entitled “FRACTURE FIXATION SYSTEM AND METHOD”; U.S. patent application Ser. No. 12/044,884 filed on Mar. 7, 2008, entitled “TRANSDISCAL INTERBODY FUSION DEVICE AND METHOD”; U.S. patent application Ser. No. 12/044,880 filed on Mar. 7, 2008, entitled “SYSTEMS, METHODS AND DEVICES FOR SOFT TISSUE ATTACHMENT TO BONE”; U.S. patent application Ser. No. 12/024,938 filed on Feb. 1, 2008, entitled “SYSTEMS, DEVICES AND METHODS FOR STABILIZING BONE”; and U.S. patent application Ser. No. 12/025,537 filed on Feb. 4, 2008, entitled “METHODS AND DEVICES FOR STABILIZING BONE COMPATIBLE FOR USE WITH BONE SCREWS”. All of these patent applications are incorporated herein by reference in their entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD OF THE INVENTION

The invention relates to devices, systems and methods for treating and supporting bone, including bone within vertebral bodies suffering from a vertebral compression fracture (VCF). More particularly, the devices, methods and systems described herein relate to rotary handles and applicator systems and controls for inserting self-expanding bone support implants.

### BACKGROUND OF THE INVENTION

Deterioration of bone tissue, and particularly micro-architecture deterioration, can result from a variety of factors including disease, aging, stress and use. For example, osteoporosis is a disease characterized by low bone mass and micro-architecture deterioration of bone tissue. Osteoporosis leads to bone fragility and an increase fracture risk. While osteoporosis affects the entire skeleton, it commonly causes fractures in the spine and hip. Spinal or vertebral fractures have serious consequences, with patients suffering from loss of height, deformity, and persistent pain that can significantly impair mobility and quality of life. Vertebral compression fractures (VCFs) and hip fractures are particularly debilitating and difficult to effectively treat.

Devices for supporting and repairing bone, including implants for repairing spinal compressions including VCFs have been described. One particularly useful type of implant for support and/or treatment of bone are self-expanding implants that may be deployed within bone to cut through the bone with little or any compression, and may be filled with one or more bone fillers (e.g., cement) in the regions within and around the implant for added support. Such implants may also act as supports or anchors for additional implants.

These bone implants (which are described in greater detail below) may be inserted using a controller (e.g., applicator system) that must provide support for the implant during and before implantation. For example, the implant may be released to self-expand within the bone, and must be manipulated into position and released while maintaining force on the implant to maintain it in a compressed (delivery) configuration. The inserter must allow precise control of the release of the implant into the bone. It may also be beneficial to allow the implant to be removed using the inserter.

It may be beneficial to have the inserter be modular, so that one or more portions could be reused, saving cost and time. For example, a handle portion may be re-used by connecting to various elongate (e.g., cannula) portions of the applicator.

It may also be helpful to provide a device having a minimum of components, and devices that are configured to include one or more failsafe mechanisms that permit the implant to be removed even in case the implant or applicator becomes jammed or otherwise disrupted.

Related U.S. application Ser. No. 12/024,938 (filed on Feb. 1, 2008), titled “SYSTEMS, DEVICES AND METHODS FOR STABILIZING BONE”) describes bone stabilization devices and methods for inserting them using a delivery device. The delivery device may be configured to include a cannula (or multiple cannula) and one or more trocars. As mentioned above, it would be extremely beneficial to have a delivery device including a handle that can be used to control the delivery and/or expansion of an implant device.

Examples of controllers, inserters, handles and devices forming such an improved handle are provided herein.

### SUMMARY OF THE INVENTION

Described herein are handles and applicator systems including handles for engaging delivery (and/or retrieval) of a bone stabilization device, as well as systems or kits including handles, and methods for using them.

An applicator (or applicator system) may include a handle region and an elongate linkage member that couples with the handle. In particular, described herein are rotary applicator handles that are configured to couple with the proximal end of the elongate linkage member and drive the axial motion (e.g., in the direction of the long axis of the elongate linkage member) of a portion of the elongate linkage member. An implant such as a bone stabilizing implant may be coupled to the distal end of the elongate linkage member, and axial movement of a portion of the elongate linkage member may result in expansion or contraction of the implant. As used herein, “axial” motion of a component of the elongate linkage member refers to motion in the direction of the long axis of the elongate linkage member. For example, an elongate linkage member may include a first elongate member that may move relative to a second elongate member. In some variations the first elongate member is an outer (e.g., cannula) member and the second elongate member is an inner (e.g., rod) member. The outer cannula and the inner rod may coaxially slide relative to each other, which is one type of “axial” movement. Axial movement of the elongate linkage member is translated into force across an implant that is coupled to the distal end of the elongate linkage member, causing the implant to collapse (e.g., into a narrow-diameter delivery configuration) or expand (e.g., into an expanded-diameter deployed configuration in which a plurality of struts bow out from the body of the implant).

In general, the handles described herein are rotary applicator handles that are activated by rotating a control on the handle (e.g., a knob, a rotating grip, etc.). Rotating the control drives rotation of a rotary gear within the handle, and the rotary gear drives axial movement of a portion of an elongate linkage member when an elongate linkage member is coupled to the handle. In variations in which the elongate linkage member includes a first elongate member and a second elongate member that are movable relative to each other, the proximal ends of the first and second elongate members are held in separate seats in the handle. By holding the proximal ends of the first and second elongate members, these members may be moved relative to each other, thereby controlling the motion of the implant coupled to the distal end of the elongate linkage member. Typically the implant is coupled to the distal end of the elongate linkage member so that the proximal end is connected to one of the elongate members forming the elongate linkage member (e.g., the first elongate linkage member) and the distal end of the implant is coupled to the distal end of the other elongate linkage member (e.g., the second elongate linkage member).

In some variations, the rotary applicator handles described herein are ratcheting handles in which the rotary gear is a ratcheting gear including a pawl that helps control the direction of axial movement driven by the gear. A control on the handle (e.g., a direction switch or a ratchet switch) may be used to select the direction of movement enabled by the handle. This control may be connected to the pawl. Other controls, including safety controls for releasing the force applied by the handle to the elongate linkage member (and therefore the implant), or for releasing the elongate linkage member from the handle, may also be included. For example, the handles described herein may include a control for regulating/controlling the release of the stabilization device. Stabilization devices are typically self-expanding devices, and the control may regulate the self-expansion so that the rate and degree of self-expansion allowed is regulated. The handles may be lockable, and may include a latch or other locking structure. These handles may also include ratcheting mechanism or other controlled expansion/release mechanism. In some variations the devices include a failsafe release configured to release either the applicator and/or the device. These devices may also include a one or more finger controls for controlling the handle, and the handle may be configured for gripping in one or more of the subject's hands.

In some variations, the handle includes indicators or sensors. For example, the handle may include an indicator of the orientation of the implant attached to the distal end of a coupled elongate linkage member. In particular, the handle may be configured so that the elongate linkage member is not rotated when axial motion is applied and therefore the implant is not rotated during delivery of the device. For example, the seats for the proximal end of the elongate linkage member may be keyed to prevent rotation of the implant.

The implants described herein may also be referred to as bones stabilization devices. These implants may include a self-expanding body that can be deployed in a linear configuration. The deploying configuration is typically an elongate tubular shape that is open at both ends. In some variations the device may have an elongate, substantially tubular shape that includes a plurality of struts extending along the length of the implant in the deployed configuration. For example, the struts maybe extended laterally in an expanded configuration. Expansion of the struts may foreshorten the implant. A self-reshaping (e.g., self-expanding) device may include a preset configuration that is expanded, and may reset from another configuration into the preset configuration (or vice versa). For example, the devices may include a linear configuration (a deployed configuration) and an expanded configuration. The linear configuration can be stabilized by constraints that prevent self-reshaping of the device into an anchoring (expended) configuration. Self-reshaping to an anchoring configuration may be performed by two or more linear portions of the device, which (upon release from constraint) radially-expand into bowed struts of various configurations, while at the same time shortening the overall length of the device. Embodiments of the struts may include a cutting surface on the outwardly leading edge or surface of the strut, which cuts through cancellous bone as it radially expands. After implantation within a vertebral body, the bowed struts may expand though the cancellous bone to contact the cortical bone of the inner surfaces of superior and inferior endplates of the compressed vertebral body, and push the endplates outward to restore the vertebral body to a desired height.

In general, the implants described herein may be inserted into tissue (e.g., bone such as a vertebra) so that they do not foreshorten when allowed to self-expand. As described in greater detail below, this may be accomplished by controlling both the proximal and distal ends (or end regions) of the implant with the applicator. Thus, the applicator (including the handle) may be configured to control the relative motions of the ends of the implant. For example, if the distal end is held while the proximal end is allowed to foreshorten, the device may be inserted without distally foreshortening or otherwise moving. Movement of the distal end of the device may result in the implant moving undesirably from the implantation site, and may cause damage or inaccuracy.

The implant maybe prepared for insertion by collapsing it. An applicator or inserter (described below) may be used to collapse it from a pre-biased expanded configuration, in which the struts are bowed or otherwise expended, and a more linear collapsed or delivery configuration, in which the struts are collapsed towards the body. For example, the step of delivering the first self-expanding implant may include the step of applying a restraining force across the implant to hold the first implant in a collapsed configuration. In some variations, the method also includes the step of applying a restraining force across the first implant by applying force across the implant to collapse a plurality of expandable struts along the implant.

The step of releasing restraining forces to radially expand the self-expanding implant within the cancellous bone may comprise allowing the proximal end of the implant to foreshorten. The step of releasing restraining forces to radially expand the first and second self-expanding implants within the cancellous bone may also (or alternatively) comprise removing the distal end portion of the implant for a first inserter region and removing the proximal end portion of the implant from a second inserter region.

Any of the handle devices described herein may be used with any appropriate elongate linkage member. In some variations, a handle and an elongate linkage member may be used together to form an applicator or applicator system. The handles described herein may be reusable or disposable. In some variations a handle is intended for use in with multiple implants in a single procedure; each implant may be connected to a separate elongate linkage member. Thus, in some variations the rotary applicator handles described herein are configured for use with a single size of implant; in other variations, the handle may be used or adapted for use with implants of different sizes. Handles may distinguish different sizes of implants based on the shape (e.g., the keyed shape) of the proximal end of the elongate linkage member to which the implant is attached distally. In some variations the handle distinguishes different sizes of implants based on the separation between the proximal ends of first and second elongate members forming the elongate linkage member.

Rotary applicator handles may be formed of any appropriate materials, including metals, plastics (e.g., polymeric materials), ceramics, or the like, including any combination thereof.

For example, described herein are rotary applicator handle for delivery or removal of a bone stabilizing implant that is distally coupled to an elongate linkage member. These handles may include: a handle grip configured to be held in the palm of a hand; a housing at least partially surrounding a first seat configured to hold the proximal end of a first elongate member of the elongate linkage member and a second seat configured to hold the proximal end of a second elongate member of the elongate linkage member; a rotary gear within the housing, the rotary gear configured to drive the axial motion of the first member of the elongate linkage member relative to the second member of the elongate linkage member; and a rotatable control coupled to rotary gear and configured to rotate the rotary gear.

The rotary gear may be a ratcheting gear comprising a pawl. In some variations, the rotary applicator handle includes a directional switch coupled to the rotary gear and configured to control direction of axial motion driven by the rotary gear.

In some variations, the rotary gear comprises a drive shaft. The rotary or rotatable control may be a knob that rotates the drive shaft.

The rotary applicator may also include an indicator to indicate the orientation of the bone stabilizing implant relative to the handle. The handle may be marked (e.g., alphanumerically, etc.) to indicate the size of the implant that it is to be used with. The rotary applicator handle may also include a release control configured to release the elongate linkage member from the handle. For example, the handle may include a force release control configured to release the axial force applied to the elongate linkage member by the handle.

The rotary applicator handle may include a mating region configured to mate with a shaft stabilizer on the first member of the elongate linkage member. The mating region may be at the distal end of the handle, and may be a keyed fitting, maintaining the orientation of the elongate linkage member (and therefore the implant) when engaged with the handle.

In some variations the rotatable control is a rotatable control grip. This rotatable grip may be configured for use by a second hand (e.g., separate from the hand holding the handle grip), or it may be a finger grip, so that it may be rotated by the thumb and index finger, for example.

In general, the expansion and contraction of the implant (and particularly a self-expanding implant) may be controlled. For example, when the implant is converted a (constrained) elongate, tubular delivery configuration having a small cross-section to an expanded configuration in which the struts extend from the body of the device, the implant may be foreshortened. The applicator system controls the deployment of the implant (from the compressed configuration to the expanded configuration) by applying axial force to pull apart (collapse) or draw together (expand) the proximal and distal ends of the implant. One end of the implant (e.g., the distal end) may be held relatively motionless while the applicator system moves the other end to collapse or expand the implant. Preventing the distal end from moving during expansion or collapse may prevent damage to the patient, and may help maintain the position of the implant during insertion. For example, the rotary gear may be configured to axially move the second seat relative to the first seat so that the proximal end of an implant coupled to the first member of the elongate linkage member moves while the distal end of the implant remains relatively stationary.

In some variations, the handle is a ratcheting applicator handle for delivery or removal of a bone stabilizing implant that is distally coupled to an elongate linkage member. In this example, the handle includes: a first handle grip region; a housing at least partially surrounding a first seat configured to hold the proximal end of an inner member of the elongate linkage member and a second seat configured to hold the proximal end of an outer member of the elongate linkage member; a ratcheting gear within the housing, the ratcheting gear configured to drive the axial motion of the outer member of the elongate linkage member relative to the inner member of the elongate linkage member; a rotatable grip coupled to ratcheting gear and configured to rotate the ratcheting gear; and a directional switch coupled to a pawl and configured to select the axial direction that the outer member is driven relative to the inner member.

As mentioned, any of these handles may be used as part of an inserter or applicator system. Thus, described herein are inserter systems for delivery or removal of a bone stabilizing implant that include: an elongate linkage member configured to distally couple with the bone stabilizing implant and a rotary handle. The elongate linkage member may include: a first elongate member configured to releasably couple at its distal end with the proximal end region of the bone stabilizing implant; and a second elongate member configured to releasably couple at its distal end with the distal end region of the bone stabilizing implant. The rotary handle may include: a handle grip region; a housing at least partially surrounding a first seat configured to hold the proximal end of the first elongate member and a second seat configured to hold the proximal end of the second elongate member; a rotary gear within the housing, the rotary gear configured to drive the axial motion of the first member relative to the second elongate member; and a rotatable control configured so that rotation of the rotatable control moves the rotary gear.

As mentioned, the first member may comprise an outer cannula and the second elongate member may comprise an internal rod. These outer and inner members may be coaxially arranged.

The elongate linkage member may also include an end grip at the proximal end of the first elongate member that is keyed to fit within the first seat of the rotary handle. The rotary gear may be a ratcheting gear comprising a pawl. The system may also include a directional switch coupled to the rotary gear and configured to control the direction of axial motion driven by the rotary gear.

In some variations, the system also includes a self-expanding implant. Any of the implants described herein may be used, including implants having a plurality of self-expanding struts and a proximal attachment region configured to releasably attach to the first elongate member and a distal attachment region configured to releasably attach to the second elongate member.

Also described herein are methods of using the rotary handles described. For example, a method of collapsing and expanding a self-expanding implant is described. This method may include the steps of: seating the proximal end of an elongate linkage member within a rotary applicator handle so that the proximal end of a first elongate member of the elongate linkage member is held within a first seat and the proximal end of a second elongate member of the elongate linkage member is held within a second seat; and rotating a control on the rotary applicator handle to drive a rotary gear that axially moves the first elongate member relative to the second elongate member so that the proximal end of a self-expanding implant that is coupled to the distal end of the first elongate member is moved relative to the distal end of the self-expanding implant that is coupled to the distal end of the second elongate member.

The step of rotating the control on the rotary applicator handle may include limiting the axial motion of the first elongate member relative to the second elongate member to prevent damage to the self-expanding implant. A limiter may be included as a stop of other structure within the handle, limiting axial motion to within a specified range. This range may be adjustable in variations of the handle that are used for different sized implants.

The step of rotating the control on the rotary applicator may comprise moving the first elongate member relative to the second elongate member without substantially moving the second elongate member. As mentioned above, this may prevent movement of the distal end of the implant.

The methods may be performed with any of the ratcheting handles described. For example, the step of rotating the control on the rotary applicator handle may include driving a ratcheting rotary gear comprising a pawl. In some variations, the method may therefore include the step of selecting the direction of axial motion by switching a ratchet switch that is coupled to a pawl.

The method may also include the steps of releasing the device from the applicator system. For example the method may include the steps of disengaging (e.g., rotating) the first and second members to release the proximal and distal ends of the implant from the elongate linkage member. This step may be performed in some variations while the elongate linkage member is attached to the handle, or after the two are decoupled. For example, the method may include the steps of activating a control on the rotary applicator handle to release the axial force applied to the elongate linkage member by the rotary applicator handle. In some variations, the method may also include the steps of releasing the elongate linkage member from the handle.

### BRIEF DESCRIPTION OF THE DRAWINGS

FIG. 1 shows one variation of a system including a self-expanding bone support implant and an applicator.

FIGS. 2A-2E are variations of stabilization devices.

FIGS. 3A and 3B are enlarged side and side perspective views (respectively) of the stabilization device shown in FIG. 2A.

FIGS. 4A and 4B are enlarged side and side perspective views (respectively) of the stabilization device shown in FIG. 2C.

FIGS. 5A and 5B are enlarged side and side perspective views (respectively) of the stabilization device shown in FIG. 2E.

FIG. 6A is one variation of a stabilization device having a plurality of continuous curvature of bending struts removably attached to an inserter.

FIG. 6B is another variation of a stabilization device removably attached to an inserter.

FIG. 7A is another variation of a stabilization device connected to an inserter. FIGS. 7B and 7C show detail of the distal and proximal ends (respectively) of the stabilization device and inserter of FIG. 7A.

FIG. 8A is one variation of a handle that may be used with an inserter.

FIGS. 8B-8E illustrate connecting an inserter to a handle such as the handle of FIG. 8A.

FIGS. 9A-9D illustrate the operation of an inserter and handle in converting a stabilization device from a relaxed, deployed configuration (in FIGS. 9A and 9B) to a contracted, delivery configuration (in FIGS. 9C and 9D).

FIG. 10 is one variation of an inserter connected to a stabilization device within an access cannula.

FIG. 11 shows one variation of a trocar and access cannula.

FIG. 12A-12C shows one variation of a hand drill.

FIG. 13 shows one variation of a cement cannula and two cement filling devices.

FIGS. 14A-14D show different variations of an access cannula that may be used with a stabilization device and inserter, trocar, drill, and cement cannula, respectively.

FIGS. 15A-15G illustrate one method of treating a bone.

FIGS. 16A-16B illustrate one method of using bone cement with the stabilization devices described herein.

FIG. 16C shows two implanted stabilization device and pedicle screws.

FIGS. 17A-17D show a series of lateral views of a vertebral body with a height HI (anterior on the left, posterior on the right) at a cross-section along a sagittal plane near a pedicle, showing (FIG. 17A) insertion of a deployment device into a drilled channel, an expandable vertebral body stabilization device contained within the deployment device.

FIG. 17B shows an early point in the deployment of a self-reshaping vertebral stabilization device, with expandable struts beginning to expand.

FIG. 17C shows full expansion of the expandable struts of the self-reshaping device and consequent restoration of vertebral body to a height H2.

FIG. 17D shows injection of a stabilizing composition into the space within the expanded struts of the self-reshaping device and into available space surrounding the device.

FIGS. 18A-18C illustrates another variation of a stabilization device.

FIG. 19A shows one variation of a handle for an applicator; FIG. 19B shows another variation of a handle for an applicator.

FIG. 20A shows another variation of a handle for an applicator.

FIG. 20B shows one variation of an elongate linkage member portion of an applicator.

FIGS. 21A and 21B show front and back exploded views, respectively of a handle such as the handle shown in FIG. 19A.

FIGS. 22A-22C illustrate various components of a handle as described.

FIGS. 23A and 23B illustrate another variation of an applicator.

FIG. 23C shows the handle region of the applicator shown in FIG. 23A.

FIGS. 24A and 24B show isometric and side perspective views, respectively, of a handle portion of an applicator.

FIGS. 25A-25J show a handle such as the handle shown in FIGS. 24A and 24B in which component parts of the handle are sequentially removed to illustrate the connection between the different functional components.

FIGS. 26A and 26B show front and isometric perspective views, respectively, of another applicator including a handle and elongate linkage member.

FIG. 27A shows a back view of the handle of the device shown in FIGS. 26A and 26B.

FIG. 27B shows a side perspective view of the handle of FIG. 27A.

FIGS. 28A and 28B illustrate one variation of an elongate linkage member of an applicator.

FIG. 29 illustrates interaction of the handle and elongate linkage member of an applicator such as the one shown in FIG. 26A.

FIG. 30 shows an exploded view of the handle of the applicator shown in FIG. 26A.

### DETAILED DESCRIPTION OF THE INVENTION

The devices, systems and methods described herein may aid in the treatment of fractures and microarchitetcture deterioration of bone tissue, including vertebral compression fractures (“VCFs”). The implantable stabilization devices described herein (which may be referred to as “implants,” “stabilization devices,” or simply “devices”) may help restore and/or augment bone. Thus, the stabilization devices described herein may be used to treat pathologies or injuries. For purposes of illustration, many of the devices, systems and methods described herein are shown with reference to the spine. However, these devices, systems and methods may be used in any appropriate body region, particularly bony regions. For example, the methods, devices and systems described herein may be used to treat hip bones.

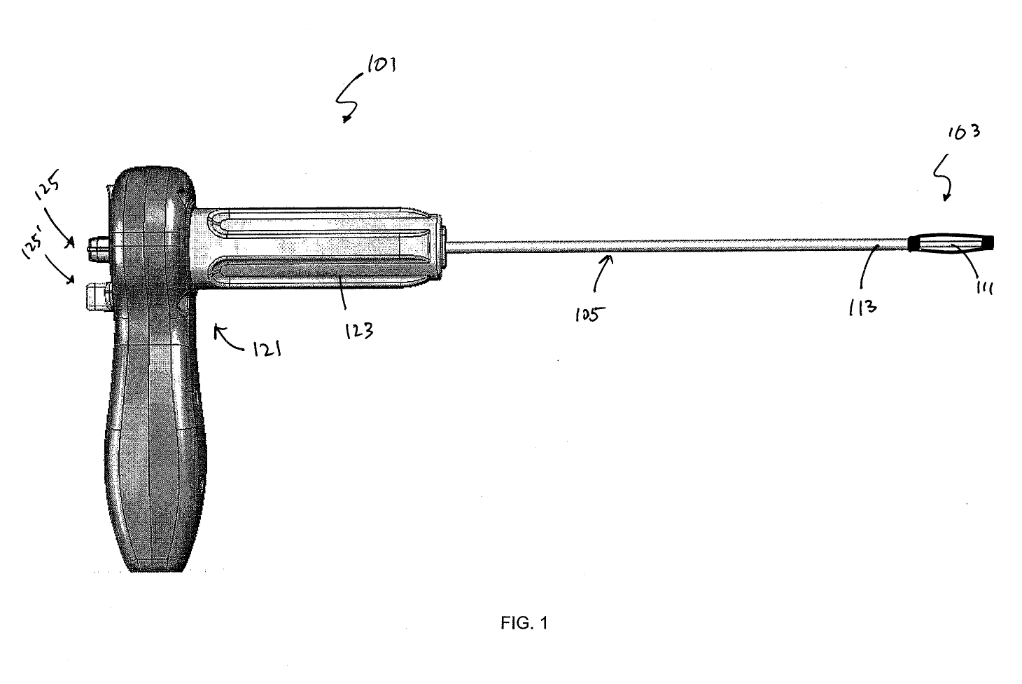

In general, the devices and systems described are rotary handles and systems including rotary handles for the insertion and/or removal of one or more bone stabilization devices. The systems may also be referred to as applicators or applicator systems. An applicator may include a handle and an elongate cannula region. An example of one variation of a system including an applicator and a bone stabilization device is shown in FIG. 1. In FIG. 1, the applicator 101 includes a handle portion 107 and an elongate cannula 105, which may be referred to as a delivery device or as an elongate linkage member. An implant 103 is attached to the distal end of the applicator 101. In this example, the implant is held in a collapsed configuration by applying force from both ends of the implant. In this example, the elongate linkage member includes an inner member (rod) 111 and an outer member 113 that are movably (slideably) disposed relative to each other. This variation is described in greater detail below. In FIG. 1, the proximal end of the bone stabilization device is releasably coupled to the outer member 113 and the distal end of the implant is releasably coupled to the inner member 111. The applicator 101 may separately control the relative motion of the proximal and distal end of the implant (which is pre-biased to self-expand to a delivery configuration) by controlling the relative motions of the outer cannula 113 and the inner member 111 at the handle 121. In this example, the handle includes a ratchet mechanism 123 (e.g., a rotary gear including a pawl, not visible in FIG. 1) and a number of controls 125,125′ for directing the motion of the applicator.

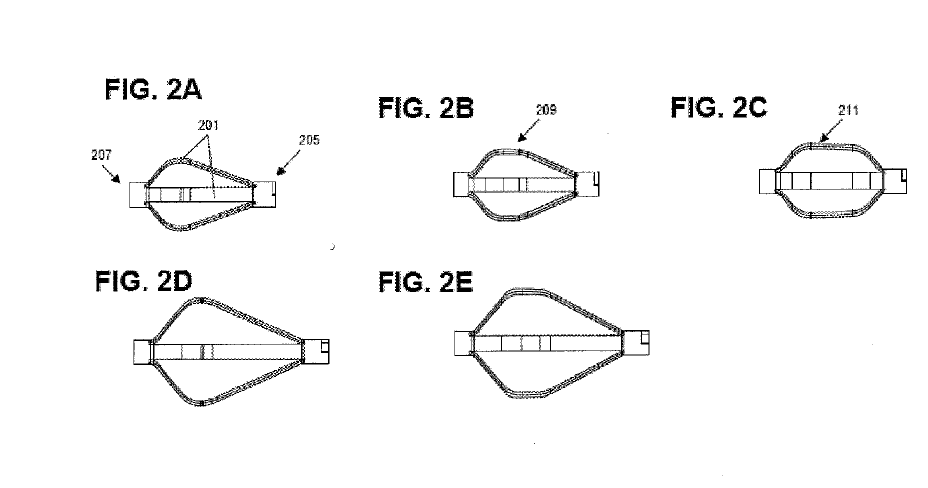

Any of the applicators or inserters described herein may be used with any appropriate bone stabilization device (typically referred to as a “stabilization device”), examples of which are provided herein. These stabilization devices may be a self-expanding device that expands from a compressed profile having a relatively narrow diameter (e.g., a delivery configuration) into an expanded profile (e.g., a deployed configuration). Stabilization devices generally include a shaft region having a plurality of struts that may extend from the shaft body. The distal and proximal regions of a stabilization device may include one or more attachment regions configured to attach to an inserter for inserting (and/or removing) the stabilization device from the body. FIGS. 2A through 6 and 18A-C show exemplary stabilization devices.

Side profile views of five variations of stabilization devices are shown in FIGS. 2A through 2E. FIG. 2A shows a 10 mm asymmetric stabilization device in an expanded configuration. The device has four struts 201, 201′, formed by cutting four slots down the length of the shaft. In this example, the elongate expandable shaft has a hollow central lumen, and a proximal end 205 and a distal end 207. By convention, the proximal end is the end closest to the person inserting the device into a subject, and the distal end is the end furthest away from the person inserting the device.

The struts 201, 201′ of the elongate shaft is the section of the shaft that projects from the axial (center) of the shaft. Three struts are visible in each of FIGS. 2A-2E. In general, each strut has a leading exterior surface that forms a cutting surface adapted to cut through cancellous bone as the strut is expanded away from the body of the elongate shaft. This cutting surface may be shaped to help cut through the cancellous bone (e.g., it may have a tapered region, or be sharp, rounded, etc.). In some variations, the cutting surface is substantially flat.

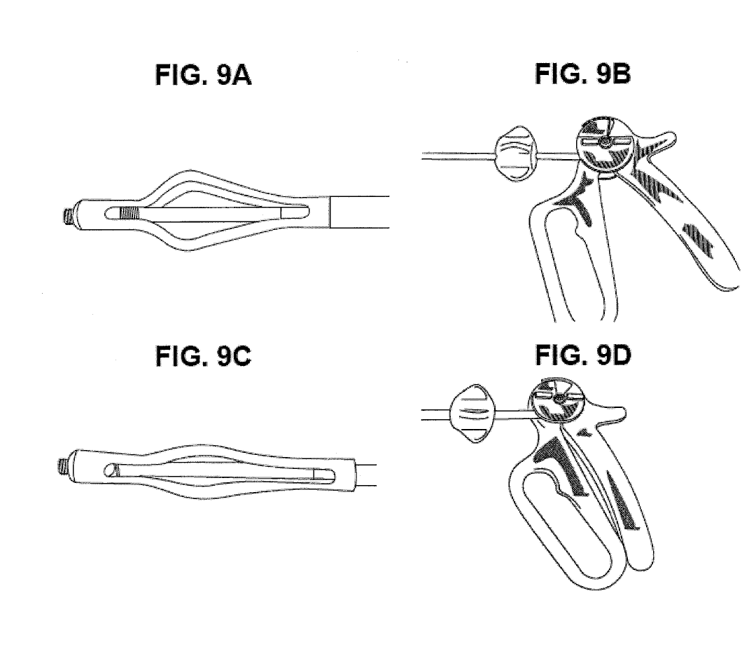

The stabilization device is typically biased so that it is relaxed in the expanded or deployed configuration, as shown in FIGS. 2A to 2E. In general, force may be applied to the stabilization device so that it assumes the narrower delivery profile, described below (and illustrated in FIG. 9C). Thus, the struts may elastically bend or flex from the extended configuration to the unextended configuration.

The struts in all of these examples are continuous curvature of bending struts. Continuous curvature of bending struts are struts that do not bend from the extended to an unextended configuration (closer to the central axis of the device shaft) at a localized point along the length of the shaft. Instead, the continuous curvature of bending struts are configured so that they translate between a delivery and a deployed configuration by bending over the length of the strut rather than by bending at a discrete portion (e.g., at a notch, hinge, channel, or the like). Bending typically occurs continuously over the length of the strut (e.g., continuously over the entire length of the strut, continuously over the majority of the length of the strut (e.g., between 100-90%, 100-80%, 100-70%, etc.), continuously over approximately half the length of the strut (e.g., between about 60-40%, approximately 50%, etc.).

The “curvature of bending” referred to by the continuous curvature of bending strut is the curvature of the change in configuration between the delivery and the deployed configuration. The actual curvature along the length of a continuous curvature of bending strut may vary (and may even have “sharp” changes in curvature). However, the change in the curvature of the strut between the delivery and the deployed configuration is continuous over a length of the strut, as described above, rather than transitioning at a hinge point. Struts that transition between delivery and deployed configurations in such a continuous manner may be stronger than hinged or notched struts, which may present a pivot point or localized region where more prone to structural failure.

Thus, the continuous curvature of bending struts do not include one or more notches or hinges along the length of the strut. Two variations of continuous curvature of bending struts are notchless struts and/or hingeless struts. In FIG. 2A, the strut 201 bends in a curve that is closer to the distal end of the device than the proximal end (making this an asymmetric device). In this example, the maximum distance between the struts along the length of device is approximately 10 mm in the relaxed (expanded) state. Thus, this may be referred to as a 10 mm asymmetric device.

FIG. 2B shows another example of a 10 mm asymmetric device in which the curve of the continuous curvature of bending strut has a more gradual bend than the devices shown in FIG. 2A. This variation may be particularly useful when the device is used to support non-cancellous bone in the deployed state. For example, the flattened curved region 209 of the continuous curvature of bending strut may provide a contact surface to support the non-cancellous bone. For example, the leading edge of the strut (the cutting edge) may expand through the cancellous bone and abut the harder cortical bone forming the exterior shell of the bony structure. FIG. 2C shows a symmetric 10 mm device in which this concept 211 is even more fully developed. FIGS. 2D and 2E are examples of 18 mm devices similar to the 10 mm devices shown in FIGS. 2A and 2B, respectively.

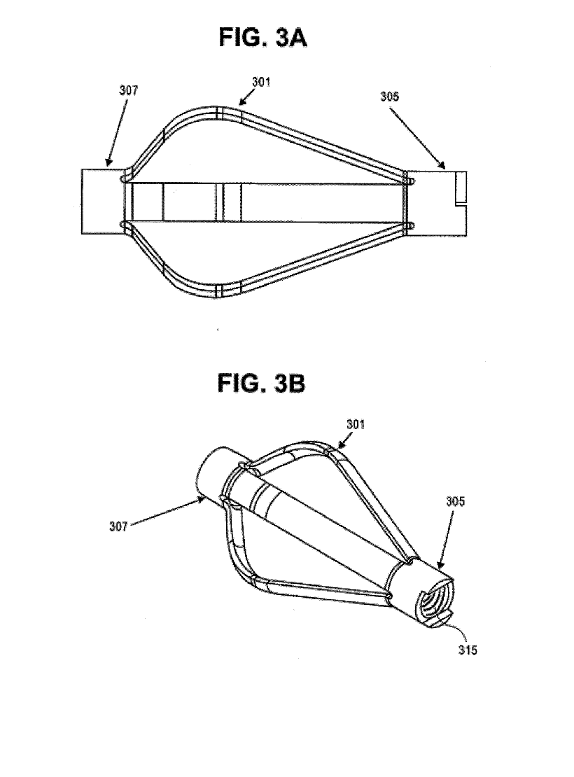

FIGS. 3A and 3B show enlarged side and side perspective views (respectively) of the 10 mm asymmetric device shown in FIG. 2A. These figures help further illustrate the continuous curve of the continuous curvature of bending strut 301. The proximal end (the end facing to the right in FIGS. 3A and 3B), shows one variation of an attachment region to which the device may be attached to one portion of an introducer. In this example, the end includes a cut-out region 305, forming a seating area into which a complementary attachment region of an inserter may mate. Although not visible in FIGS. 3A and 3B, the distal region 307 of the device may also include an attachment region. In some variations, the inner region (and/or outer region) of the proximal end 315 of the device may be threaded. Threads may also be used to engage the inserter at the proximal (and/or distal) ends of the device as part of the attachment region.

An attachment region may be configured in any appropriate way. For example, the attachment region may be a cut-out region (or notched region), including an L-shaped cut out, an S-shaped cut out, a J-shaped cut out, or the like, into which a pin, bar, or other structure on the inserter may mate. In some variations, the attachment region is a threaded region which may mate with a pin, thread, screw or the like on the inserter. In some variations, the attachment region is a hook or latch. The attachment region may be a hole or pit, with which a pin, knob, or other structure on the inserter mates. In some variations, the attachment region includes a magnetic or electromagnetic attachment (or a magnetically permeable material), which may mate with a complementary magnetic or electromagnet region on the inserter. In each of these variations the attachment region on the device mates with an attachment region on the inserter so that the device may be removably attached to the inserter.

The attachment region on the implant may be formed of a material forming the majority of the implant (e.g., a shape memory material such as a shape memory alloy), or it may be formed of a different material and secured to the rest of the implant. In particular, when the implant attachment regions comprises threads, it may be particularly advantageous to form the threads in anther material (e.g., PMMA or other polymers, ceramics, or metals) that is then secured to the shape memory alloy forming the body of the implant. In some variations the attachment regions comprise an internal threaded region at the distal end of the implant and an external threaded region at the proximal end of the implant (counter-threaded as described below). It is known that shape memory materials such as Nitinol are particularly difficult to cut threads in and to weld to, particularly in an internal diameter such as the distal end of the device. Thus, in some variations the distal end of the device includes a plug formed of PMMA or other biocompatible material that forms threads and can be inserted into the implants distal end.

The stabilization devices described herein generally have two or more releasable attachment regions for attaching to an inserter. For example, a stabilization device may include at least one attachment region at the proximal end of the device and another attachment region at the distal end of the device. This may allow the inserter to apply force across the device (e.g., to pull the device from the expanded deployed configuration into the narrower delivery configuration), as well as to hold the device at the distal end of the inserter. However, the stabilization devices may also have a single attachment region (e.g., at the proximal end of the device). In this variation, the more distal end of the device may include a seating region against which a portion of the inserter can press to apply force to change the configuration of the device. In some variations of the self-expanding stabilization devices, the force to alter the configuration of the device from the delivery to the deployed configuration comes from the material of the device itself (e.g., from a shape-memory material), and thus only a single attachment region (or one or more attachment region at a single end of the device) is necessary.

In variations of the stabilization device that include a proximal releasable attachment site and a distal releasable attachment site (which may be located at either at the proximal and distal ends, or spaced from the ends), the releasable attachment sites may be configured to operate in opposite directions. For example, when the attachment sites are threaded regions (e.g., FIG. 3A-3B), the threads on the proximal attachment or coupling site may be configured to run counterclockwise while threads on the distal attachment or coupling site are configured to run clockwise. Thus, each end of the implant may be coupled or de-coupled to the applicator may rotating in opposite directions relative to each other. In addition, the coupling regions may be configured so that the rotational tolerances are controlled so that there is very little slippage between the applicator and the implant when rotating to engage or disengage.

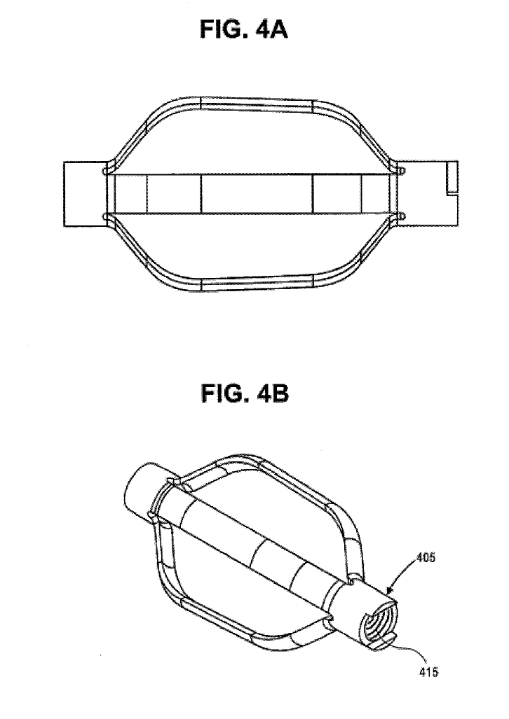

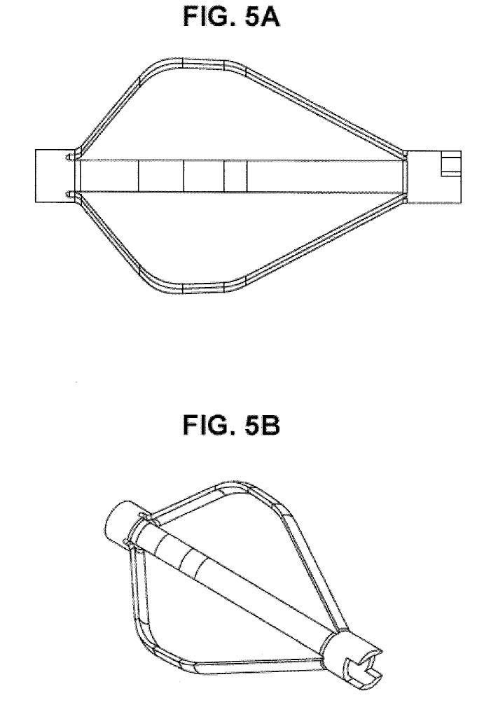

Similar to FIGS. 3A and 3B, FIGS. 4A and 4B show side and side perspective views of exemplary symmetric 10 mm devices, and FIGS. 5A and 5B show side and side perspective views of 18 mm asymmetric devices.

The continuous curvature of bending struts described herein may be any appropriate dimension (e.g., thickness, length, width), and may have a uniform cross-sectional thickness along their length, or they may have a variable cross-sectional thickness along their length. For example, the region of the strut that is furthest from the tubular body of the device when deployed (e.g., the curved region 301 in FIGS. 3A and 3B) may be wider than other regions of the strut, providing an enhanced contacting surface that abuts the non-cancellous bone after deployment.

The dimensions of the struts may also be adjusted to calibrate or enhance the strength of the device, and/or the force that the device exerts to self-expand. For example, thicker struts (e.g., thicker cross-sectional area) may exert more force when self-expanding than thinner struts. This force may also be related to the material properties of the struts.

As mentioned, in some variations, different struts on the device may have different widths or thicknesses. In some variations, the same strut may have different widths of thicknesses along its length. Controlling the width and/or thickness of the strut may help control the forces applied when expanding. For example, controlling the thickness may help control cutting by the strut as it expands.

Similarly, the width of the strut (including the width of the outward-facing face of the strut) may be controlled. The outward-facing face may include a cutting element (e.g., a sharp surface) along all or part of its width, as mentioned.

Varying the width, thickness and cutting edge of the struts of a device may modulate the structural and/or cutting strength of the strut. This may help vary or control the direction of cutting. Another way to control the direction of cutting is to modify the pre-biased shape. For example, the expanded (pre-set) shape of the struts may include one or more struts having a different shape than the other struts. For example, one strut may be configured to expand less than the other struts, or more than other struts. Thus, in some variations, the shape of the expanded implant may have an asymmetric shape, in which different struts have different expanded configurations.

The struts may be made of any appropriate material. In some variations, the struts and other body regions are made of substantially the same material. Different portions of the stabilization device (including the struts) may be made of different materials. In some variations, the struts may be made of different materials (e.g., they may be formed of layers, and/or of adjacent regions of different materials, have different material properties). The struts may be formed of a biocompatible material or materials. It may be beneficial to form struts of a material having a sufficient spring constant so that the device may be elastically deformed from the deployed configuration into the delivery configuration, allowing the device to self-expand back to approximately the same deployed configuration. In some variation, the strut is formed of a shape memory material that may be reversibly and predictably converted between the deployed and delivery configurations. Thus, a list of exemplary materials may include (but is not limited to): biocompatible metals, biocompatible polymers, polymers, and other materials known in the orthopedic arts. Biocompatible metals may include cobalt chromium steel, surgical steel, titanium, titanium alloys (such as the nickel titanium alloy Nitinol), tantalum, tantalum alloys, aluminum, etc. Any appropriate shape memory material, including shape memory alloys such as Nitinol may also be used.

Other regions of the stabilization device may be made of the same material(s) as the struts, or they may be made of a different material. Any appropriate material (preferably a biocompatible material) may be used (including any of those materials previously mentioned), such as metals, plastics, ceramics, or combinations thereof. In variations where the devices have bearing surfaces (i.e. surfaces that contact another surface), the surfaces may be reinforced. For example, the surfaces may include a biocompatible metal. Ceramics may include pyrolytic carbon, and other suitable biocompatible materials known in the art. Portions of the device can also be formed from suitable polymers include polyesters, aromatic esters such as polyalkylene terephthalates, polyamides, polyalkenes, poly(vinyl) fluoride, PTFE, polyarylethyl ketone, and other materials. Various alternative embodiments of the devices and/or components could comprise a flexible polymer section (such as a biocompatible polymer) that is rigidly or semi rigidly fixed.

The devices (including the struts), may also include one or more coating or other surface treatment (embedding, etc.). Coatings may be protective coatings (e.g., of a biocompatible material such as a metal, plastic, ceramic, or the like), or they may be a bioactive coating (e.g., a drug, hormone, enzyme, or the like), or a combination thereof. For example, the stabilization devices may elute a bioactive substance to promote or inhibit bone growth, vascularization, etc. In one variation, the device includes an elutible reservoir of bone morphogenic protein (BMP).

As previously mentioned, the stabilization devices may be formed about a central elongate hollow body. In some variations, the struts are formed by cutting a plurality of slits long the length (distal to proximal) of the elongate body. This construction may provide one method of fabricating these devices, however the stabilization devices are not limited to this construction. If formed in this fashion, the slits may be cut (e.g., by drilling, laser cutting, etc.) and the struts formed by setting the device into the deployed shape so that this configuration is the default, or relaxed, configuration in the body. For example, the struts may be formed by plastically deforming the material of the struts into the deployed configuration. In general, any of the stabilization devices may be thermally treated (e.g., annealed) so that they retain this deployed configuration when relaxed. Thermal treatment may be particularly helpful when forming a strut from a shape memory material such as Nitinol into the deployed configuration.

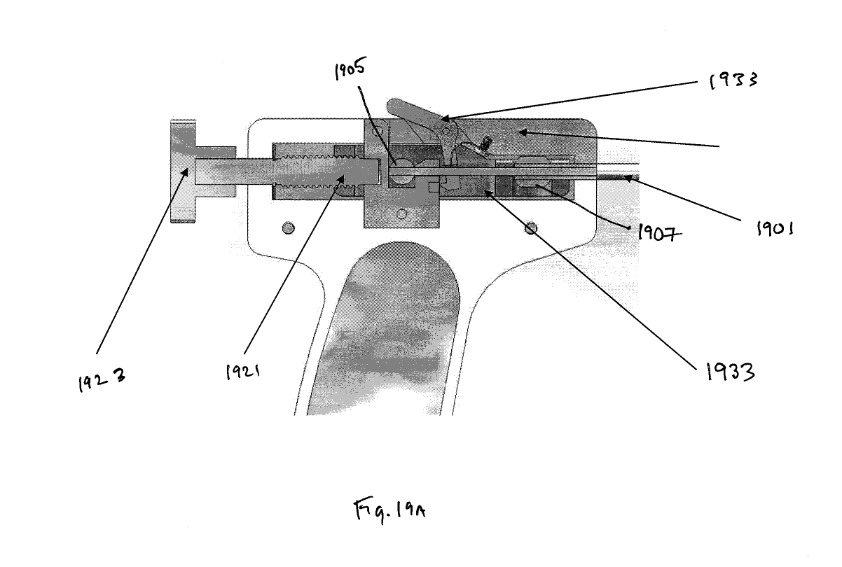

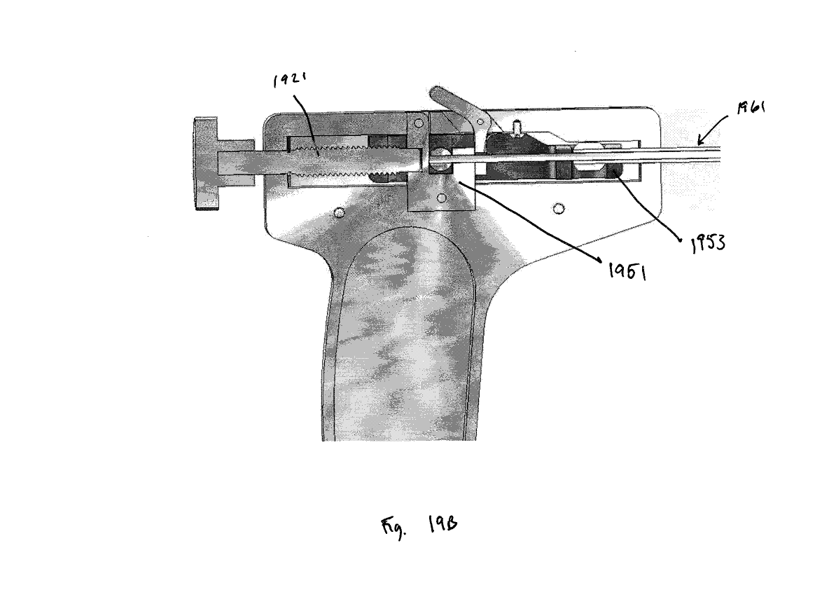

FIGS. 18A-18C illustrate another variation of a bone stabilization device. In this example, the bone stabilization device is pre-biased in an expanded configuration, and an expansion limiter is slideably coupled to the outside of the device. In general, an expansion limiter may be a tube, funnel, or other structure that may be fitted over one or both ends of the stabilization device. The stabilization device may be otherwise similar, e.g., pre-biased in the expanded configuration to those described above. The minimum diameter of the expansion limiter (which may also be referred to as an “over tube”) is typically somewhat larger than the outer diameter of the stabilization device in the collapsed configuration (prior to expansion). At least a partial length of the expansion limiter may be threaded, ratcheted, or otherwise shaped such that a relative position of the expansion controller relative to the stabilization device can be controlled and maintained. For example, at least a partial length of the exterior of the stabilization device may be shaped to mate with the expansion limiter. For example, the expansion limiter may travel on threads controlling the position of the limiter relative to the stabilization device. In this example, the position of the expansion limiter relative to the stabilization device may be changed by rotating and/or translating it. The expansion limiter may be moved along the length of the stabilization device to allow it to change diameter (e.g., expand). In variations of the device including an expansion limiter, the expansion limiter may be coupled to a member of the applicator (e.g., a first elongate member or the outer cannula member). Thus, the outer cannula member may be coupled to the limiter while the inner member is coupled to the proximal or distal end of the implant. Motion of the limiter relative to the implant may be used to expand or collapse the implant, as illustrated in FIGS. 18A-18C. As the expansion limiter 1805 in FIG. 18A is moved distally in FIGS. 18B and 18C, the implant 1801 collapses.

As mentioned, the expansion limiter may be coupled to the applicator, or may for a portion of the applicator. Thus, the applicator may move the expansion limiter relative to the stabilization device to allow it to controllably expand (preferably while leaving the distal end fixed relative to the insertion site in the body). In some variation the expansion limiter may be an outer sleeve that fits over all or a portion of the stabilization device and may be withdrawn to deliver it.

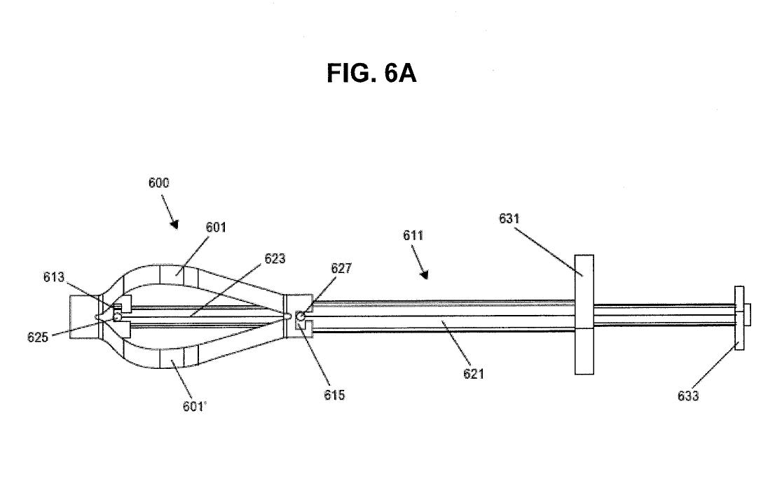

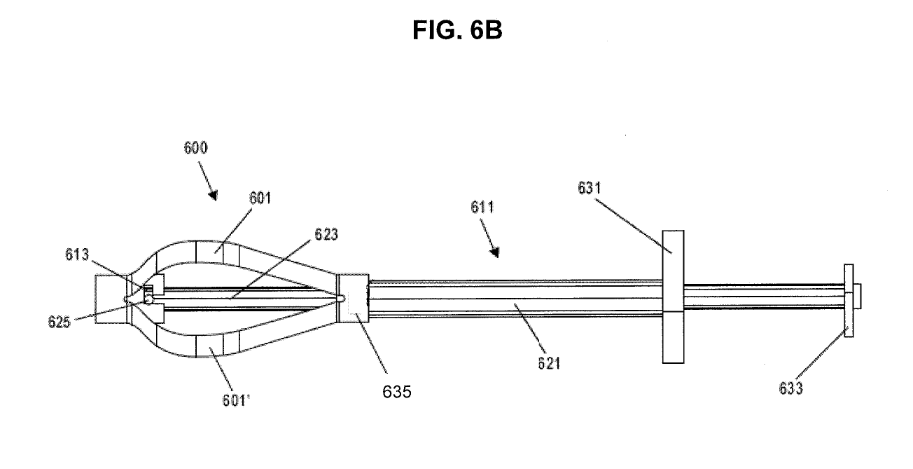

FIG. 6A shows one variation of a stabilization device 600 having a plurality of continuous curvature of bending struts 601, 601′ removably attached to an elongate linkage member (referred to here as an inserter) 611. In this example, an attachment region 615 at the proximal portion of the stabilization device is configured as an L-shaped notch, as is the attachment region 613 at the distal portion of the device. The inserter 611 in this example does not include a separate handle, although grips 631, 633 are integrally formed at the proximal end.

As mentioned, an inserter may include an elongate body having a distal end to which the stabilization device may be attached and a proximal end which may include a handle or other manipulator that coordinates converting an attached stabilization device from a delivery and a deployed configuration, and also allows a user to selectively release the stabilization device from the distal end of the inserter.

The elongate linkage member (inserter) 611 shown in FIG. 6A includes a first elongate member 621 that coaxially surrounds a second elongate member 623. In this variation, each elongate member 621, 623 includes a stabilization device attachment region at its distal end, to which the stabilization device is attached, as shown. In this example, the stabilization device attachment region includes a pin that mates with the L-shaped slots forming the releasable attachment regions on the stabilization device. In FIG. 6A the L-shaped releasable attachments on the stabilization device are oriented in opposite directions (e.g., the foot of each “L” points in opposite directions). Thus, the releasable attachment devices may be locked in position regardless of torque applied to the inserter, preventing the stabilization device from being accidentally disengaged.

The inserter shown in FIG. 6A also includes two grips 631, 633 at the proximal ends of each elongate member 621, 623. These grips can be used to move the elongate members (the first 621 or second 623 elongate member) relative to each other. The first and second elongate members of the inserter may be moved axially (e.g., may be slid along the long axis of the inserter) relative to each other, and/or they may be moved in rotation relative to each other (around the common longitudinal axis). Thus, when a stabilization device is attached to the distal end of the inserter, moving the first elongate member 621 axially with respect to the second elongate member 623 will cause the stabilization device to move between the deployed configuration (in which the struts are expanded) and the delivery configuration (in which the struts are relatively unexpanded). Furthermore, rotation of the first elongate member of the inserter relative to the second elongate member may also be used to disengage one or more releasable attachment regions of the stabilization device 613, 615 from the complementary attachment regions of the inserter 625, 627. Although he stabilization devices described herein are typically self-expanding stabilization devices, the inserter may be used with stabilization devices that do not self-expand. Even in self-expanding devices, the inserter may be used to apply additional force to convert the stabilization device between the delivery and the deployed configuration. For example, when allowed to expand in a cancellous bone, the force applied by the struts when self-expanding may not be sufficient to completely cut through the cancellous bone and/or distract the cortical bone as desired. In some variations, the inserter may also permit the application of force to the stabilization device to expand the struts even beyond the deployed configuration.

An inserter may also limit or guide the movement of the first and second elongate members, so as to further control the configuration and activation of the stabilization device. For example, the inserter may include a guide for limiting the motion of the first and second elongate members. A guide may be a track in either (or both) elongate member in which a region of the other elongate member may move. The inserter may also include one or more stops for limiting the motion of the first and second elongate members.

As mentioned above, the attachment regions on the inserter mate with the stabilization device attachments. Thus, the attachment regions of the inserter may be complementary attachments that are configured to mate with the stabilization device attachments. For example, a complimentary attachment on an inserter may be a pin, knob, or protrusion that mates with a slot, hole, indentation, or the like on the stabilization device. The complementary attachment (the attachment region) of the inserter may be retractable. For example, the inserter may include a button, slider, etc. to retract the complementary attachment so that it disconnects from the stabilization device attachment. A single control may be used to engage/disengage all of the complementary attachments on an inserter, or they may be controlled individually or in groups.

FIG. 6B is another variation of a stabilization device 600 releasably connected to an inserter 611, in which the attachment region 635 between the stabilization device and the inserter is configured as a screw or other engagement region, rather than the notch 615 shown in FIG. 6A.

In some variation the inserter includes a lock or locks that hold the stabilization device in a desired configuration. For example, the inserter may be locked so that the stabilization device is held in the delivery configuration (e.g., by applying force between the distal and proximal ends of the stabilization device). In an inserter such as the one shown in FIG. 6A, for example, a lock may secure the first elongate member to the second elongate member so that they may not move axially relative to each other.

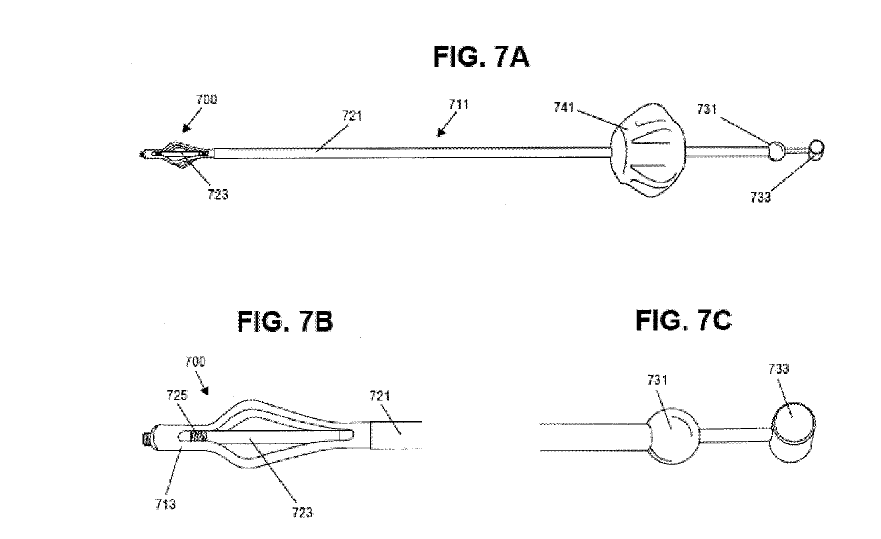

FIG. 7A is another example of an inserter 711 and an attached stabilization device 700. Similar to FIG. 6A, the stabilization device includes a first elongate member 721 attached to the proximal end of the stabilization device, and a second elongate member 723 attached to the distal end of the stabilization device. The first 721 and the second 723 elongate members are also configured coaxially (as a rod and shaft) that may be moved axially and rotationally independently of each other. The stabilization device 700 includes a plurality of continuous curvature of bending struts, shown in detail in FIG. 7B. The stabilization device 700 is shown in the deployed configuration. The distal end of the stabilization device includes a releasable attachment 713 that is configured as a threaded region which mates with a threaded complementary attachment 725 at the distal end of the structure.

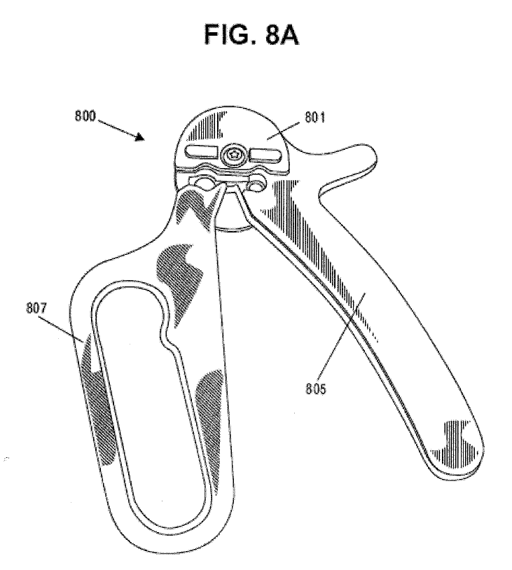

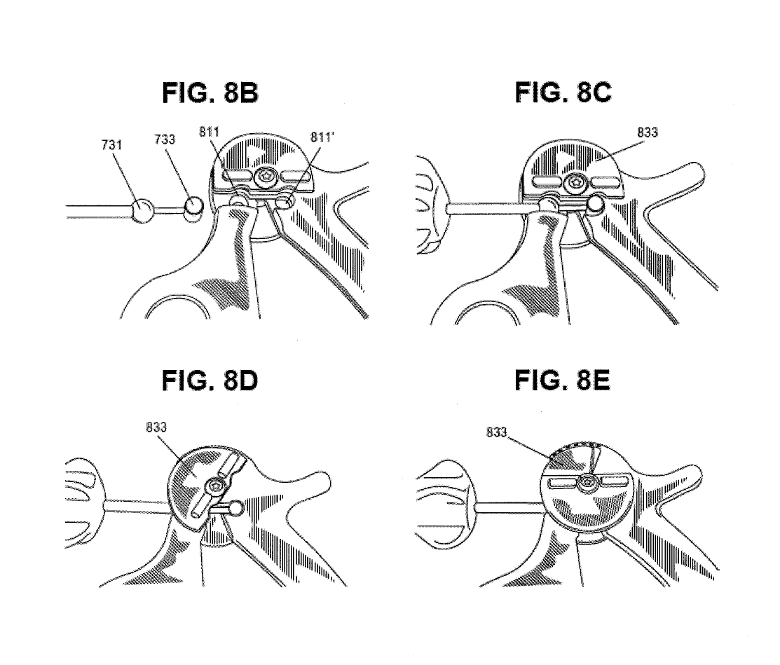

The proximal ends of the coaxial first and second elongated members 721, 723 also include grips 731, 733. These grips are shown in greater detail in FIG. 7C. As with the grips described in FIG. 6A, these grips may be grasped directly by a person (e.g., a physician, technician, etc.) using the device, or they may be connected to a handle. Thus, in some variations one or both grips are ‘keyed’ to fit into a handle, so that they can be manipulated by the handle. An example of this is shown in FIG. 8A-8E, and described below. The inserter of FIG. 7A also includes a knob 741 attached to the first elongated member 721 distal to the proximal end of the elongated member. This knob may also be used to move the first (or outer) elongate member of the inserter (e.g., to rotate it), or to otherwise hold it in a desired position. The knob may be shaped and/or sized so that it may be comfortably handheld. In some variations (described in greater detail below) this knob 741 is a keyed member that is secured to the outer member (cannula) of the inserter 711. This keyed member may be configured to secure within a handle so and may help orient the device (including the implant) and the handle, and may sever to secure the cannula in the handle. The keyed member may have an outer shape (e.g., rectangular, etc.) that locks the relative motion of all or a portion of the handle with respect to the outer member.

Any of the inserters described herein may include, or may be used with, a handle. A handle may allow a user to control and manipulate an inserter. For example, a handle may conform to a subject's hand, and may include other controls, such as triggers or the like. Thus, a handle may be used to control the relative motion of the first and second elongate members of the inserter, or to release the connection between the stabilization device and the inserter, or any of the other features of the inserter described herein.

An inserter may be packaged or otherwise provided with a stabilization device attached. Thus, the inserter and stabilization device may be packaged sterile, or may be sterilizable. In some variations, a reusable handle is provided that may be used with a pre-packaged inserter stabilization device assembly. In some variations the handle is single-use or disposable. The handle may be made of any appropriate material. For example, the handle may be made of a polymer such as polycarbonate.

FIG. 8A illustrates one variation of a handle 800 that may be used with an inserter, such as the inserter shown in FIGS. 7A-7C. The handle 800 includes a hinged joint 803, and the palm contacting 805 region and finger contacting 807 region of the handle 800 may be moved relative to each other by rotating about this hinged joint 803. This variation of a handle also includes a thumb rest 809, which may also provide additional control when manipulating an inserter with the handle. The thumb rest may also include a button, trigger, or the like.

FIGS. 8B-8E illustrate the connection of an inserter such as the inserter described above in FIGS. 7A-C into a handle 800. In FIG. 8B the proximal end of the inserter is aligned with openings 811, 811′ in the handle. These openings are configures so that the grips 731, 733 at the distal ends of the first and second elongate members of the inserter can fit into them. In this example, the grip 733 is shaped so that it can be held in the opening 811′ of the handle in an oriented fashion, preventing undesirable rotation. Thus, in FIG. 8C the proximal end of the inserter (the grips 731 and 732) are placed in the openings 811, 811′. The inserter may then be secured to the handle by rotating cover 833, as shown in FIGS. 8D and 8E.

By securing the proximal end of the inserter in the handle, the handle can then be used to controllably actuate the inserter, as illustrated in FIGS. 9A-9D. In this example the stabilization device is in the deployed configuration (shown in FIG. 9A) when the handle is “open” (shown in FIG. 9B). By squeezing the handle (rotating the finger grip region towards the palm region, as shown in FIG. 9D) the inserter applies force between the proximal and distal regions of the stabilization device, placing it in a delivery configuration, as shown in FIG. 9C.

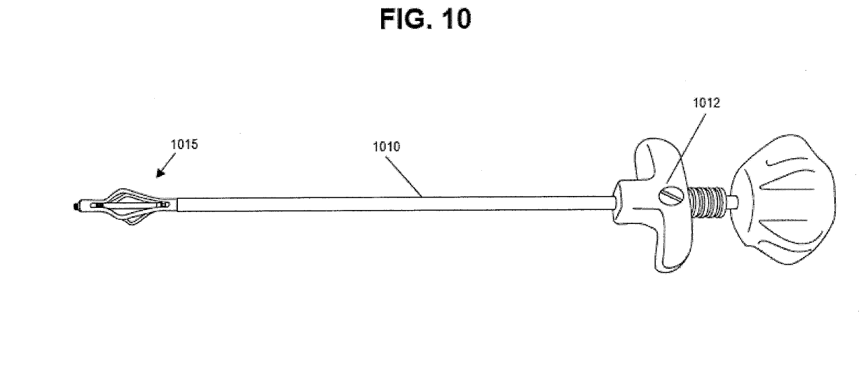

As mentioned above, in the delivery configuration the struts of the stabilization device are typically closer to the long axis of the body of the stabilization device. Thus, the device may be inserted into the body for delivery into a bone region. This may be accomplished with the help of an access cannula (which may also be referred to as an introducer). As shown in FIG. 10, the inserter 1015 is typically longer than the access cannula 1010, allowing the stabilization device to project from the distal end of the access cannula for deployment. The access cannula may also include a handle 1012.

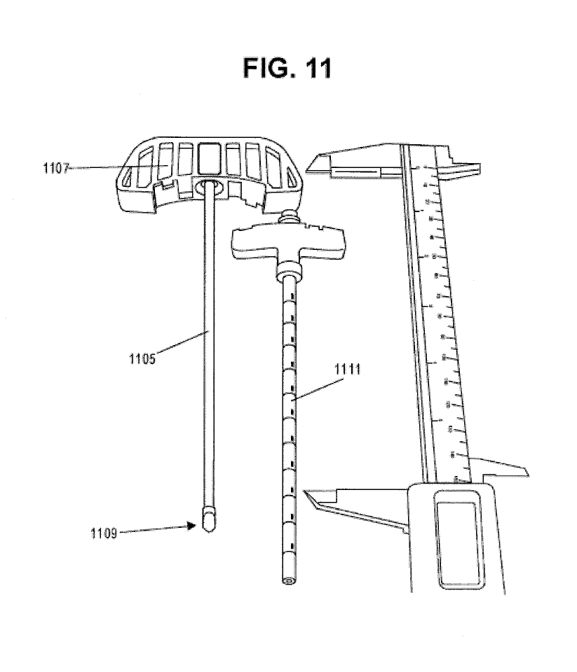

Any of the devices (stabilization devices) and applicators (including handles) may be included as part of a system or kit for correcting a bone defect or injury. FIGS. 10 through 14D illustrate different examples of tools (or variations of tools) that may be used as part of a system for repair bone. Any of these tools (or additional tools) may also be used to perform the methods of repairing bone (particularly spinal bone) described herein. For example, FIG. 11 shows a trocar 1105 having a handle 1107 and a cutting/obdurating tip 1109. This trocar 1105 may also be used with an access cannula 1111. Another example of an access cannula 1111 (or introducer) is shown adjacent to the trocar 1106 in FIG. 11. This exemplary access cannula has an inner diameter of approximately 4.2 mm, so that the trocar 1105 will fit snugly within it, and a stabilization device in a delivery configuration will also fit therein. Any appropriate length cannula and trocar may be used, so long as it is correctly scaled for use with the introducer and stabilization device. For example, the access cannula may be approximately 15.5 cm long. The trocar an introducer may be used to cut through tissue until reaching bone, so that the introducer can be positioned appropriately.

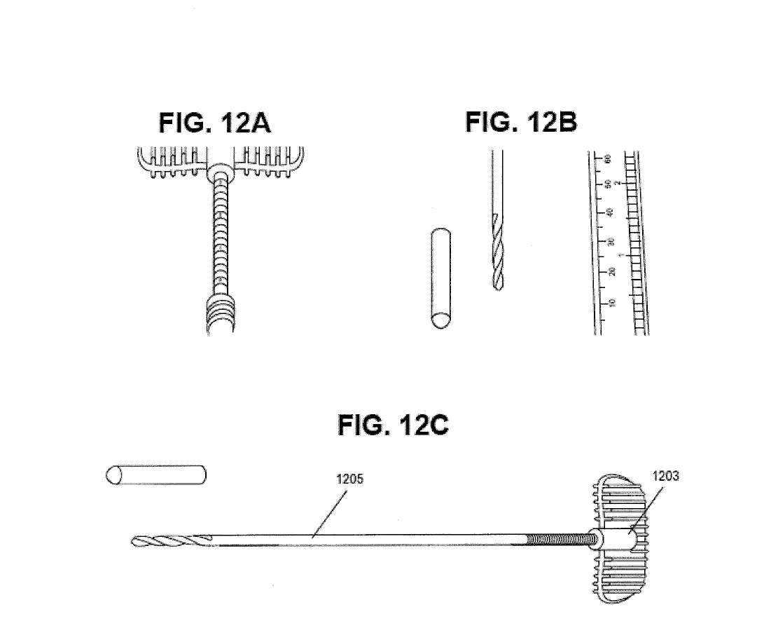

A bone drill, such as the hand drill shown in FIGS. 12A-12C, may then be used to access the cancellous bone. The twist drill 1201 shown in FIG. 12A-12C has a handle 1203 at the proximal end and a drill tip 1205 at the distal end. This twist drill may be used with the same access cannula previously described (e.g., in this example the twist drill has an outer diameter of 4.1 mm and a length of 19.5 cm). The distal (drill) end of the twist drill may extend from the cannula, and be used to drill into the bone. The proximal end of the twist drill shown in FIGS. 12A-12C is calibrated (or graduated) to help determine the distance drilled.

Any of the devices shown and described herein may also be used with a bone cement. For example, a bone cement may be applied after inserting the stabilization device into the bone, positioning and expanding the device (or allowing it to expand and distract the bone) and removing the inserter, leaving the device within the bone. Bone cement may be used to provide long-term support for the repaired bone region.

Any appropriate bone cement or filler may be used, including PMMA, bone filler or allograft material. Suitable bone filler material include bone material derived from demineralized allogenic or xenogenic bone, and can contain additional substances, including active substance such as bone morphogenic protein (which induce bone regeneration at a defect site). Thus materials suitable for use as synthetic, non-biologic or biologic material may be used in conjunction with the devices described herein, and may be part of a system includes these devices. For example, polymers, cement (including cements which comprise in their main phase of microcrystalline magnesium ammonium phosphate, biologically degradable cement, calcium phosphate cements, and any material that is suitable for application in tooth cements) may be used as bone replacement, as bone filler, as bone cement or as bone adhesive with these devices or systems. Also included are calcium phosphate cements based on hydroxylapatite (HA) and calcium phosphate cements based on deficient calcium hydroxylapatites (CDHA, calcium deficient hydroxylapatites). See, e.g., U.S. Pat. No. 5,405,390 to O'Leary et al.; U.S. Pat. No. 5,314,476 to Prewett et al.; U.S. Pat. No. 5,284,655 to Bogdansky et al.; U.S. Pat. No. 5,510,396 to Prewett et al.; U.S. Pat. No. 4,394,370 to Jeffries; and U.S. Pat. No. 4,472,840 to Jeffries, which describe compositions containing demineralized bone powder. See also U.S. Pat. No. 6,340,477 to Anderson which describes a bone matrix composition. Each of these references is herein incorporated in their entirely.

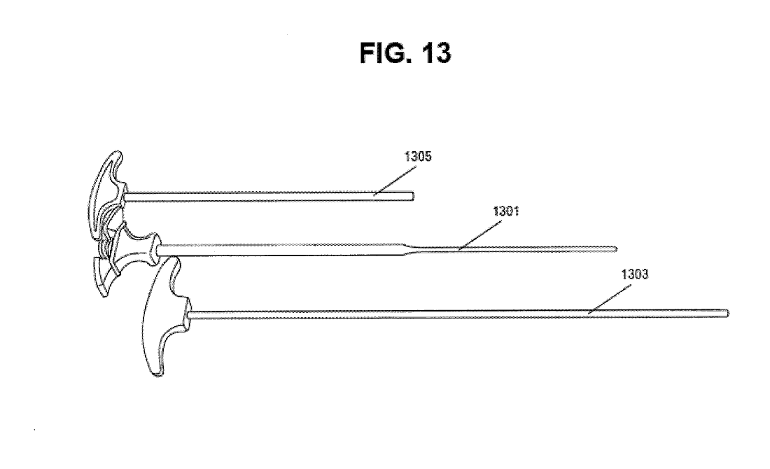

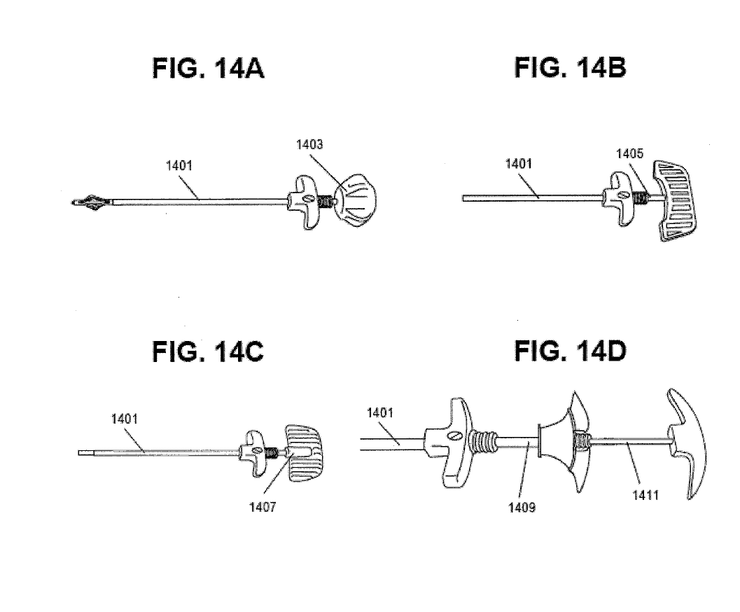

FIG. 13 shows a tapered cement cannula 1301 that may be used to deliver bone cement to the insertion site of the device, and also shows two cement obturators 1303, 1305 for delivering the cement (piston-like). The cannula delivering cement is also designed to be used through the access cannula, as are all of the components described above, including the stabilization device and inserter, trocar, and drill. This is summarized in FIGS. 14A-14D. FIG. 14A illustrates an access cannula 4101 with a stabilization device 1403 and inserter inserted through the access cannula, as shown in FIG. 10. FIG. 14B shows a trocar 1405 within the access cannula 1401. FIG. 14C shows a hand drill 1407 within the same access cannula 1401, and FIG. 14D shows a cement cannula 1409 and a cement obturator 1411 within the same access cannula 1401. These devices may be used to repair a bone.

### Exemplary Method of Repairing a Bone

As mentioned above, any of the devices described herein may be used to repair a bone. A method of treating a bone using the devices describe herein typically involves delivering a stabilization device (e.g., a self-expanding stabilization device as described herein) within a cancellous bone region, and allowing the device to expand within the cancellous bone region so that a cutting surface of the device cuts through the cancellous bone.

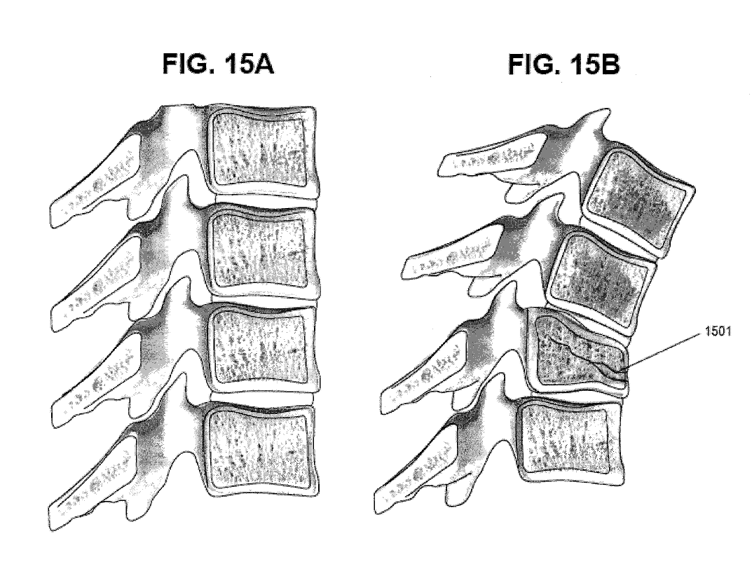

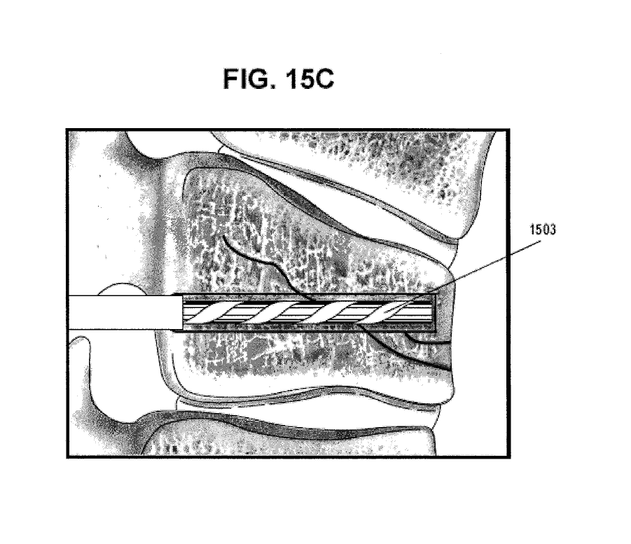

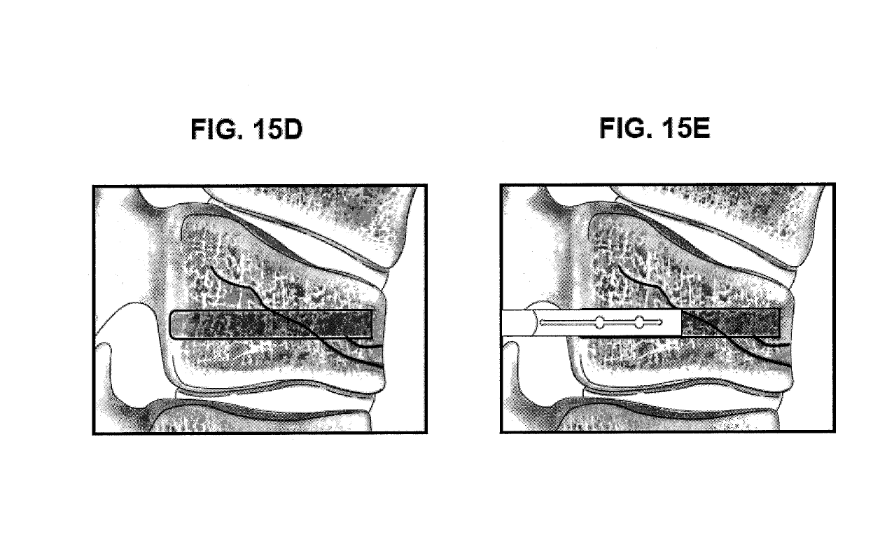

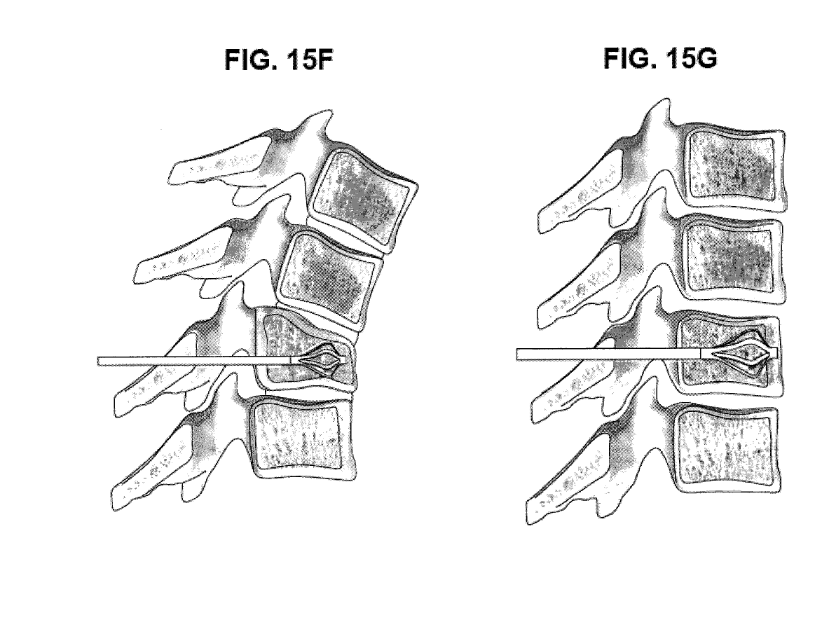

For example, the stabilization devices described herein may be used to repair a compression fracture in spinal bone. This is illustrated schematically in FIGS. 15A-15G. FIG. 15A shows a normal thoracic region of the spine in cross-section along the sagital plane. The spinal vertebras are aligned, distributing pressure across each vertebra. FIG. 15B shows a similar cross-section through the spine in which there is a compression fracture in the 11^{th} thoracic vertebra 1501. The 11^{th} vertebra is compressed in the fractured region. It would be beneficial to restore the fractured vertebra to its uninjured position, by expanding (also referred to as distracting) the vertebra so that the shape of the cortical bone is restored. This may be achieved by inserting and expanding one of the stabilization devices described herein. In order to insert the stabilization device, the damaged region of bone must be accessed.

As mentioned above, an introducer (or access cannula) and a trocar, such as those shown in FIG. 11 may be used to insert the access cannula adjacent to the damaged bone region. Any of the steps described herein may be aided by the use of an appropriate visualization technique. For example, a fluoroscope may be used to help visualize the damaged bone region, and to track the p of inserting the access cannula, trocar, and other tools. Once the access cannula is near the damaged bone region, a bone drill may be used to drill into the bone, as shown in FIG. 15C.

In FIG. 15C the drill 1503 enters the bone from the access cannula. The drill enters the cancellous bony region within the vertebra. After drilling into the vertebra to provide access, the drill is removed from the bone and the access cannula is used to provide access to the damaged vertebra, as shown, by leaving the access cannula in place, providing a space into which the stabilization device may be inserted in the bone, as shown in FIG. 15D. In FIG. 15E a stabilization device, attached to an inserter and held in the delivery configuration, is inserted into the damaged vertebra.

Once in position within the vertebra, the stabilization device is allowed to expand (by self-expansion) within the cancellous bone of the vertebra, as shown in FIG. 15F. In some variations, the device may fully expand, cutting through the cancellous bone and pushing against the cortical bone with a sufficient restoring force to correct the compression, as shown in FIG. 15G. However, in some variations, the force generated by the device during self-expansion is not sufficient to distract the bone, and the inserter handle may be used (e.g., by applying force to the handle, or by directly applying force to the proximal end of the inserter) to expand the stabilization device until the cortical bone is sufficiently distracted.

Once the stabilization device has been positioned and is expanded, it may be released from the inserter. In some variations, it may be desirable to move or redeploy the stabilization device, or to replace it with a larger or smaller device. If the device has been separated from the inserter (e.g., by detaching the removable attachments on the stabilization device from the cooperating attachments on the inserter), then it may be reattached to the inserter. Thus, the distal end of the inserter can be coupled to the stabilization device after implantation. The inserter can then be used to collapse the stabilization device back down to the delivery configuration (e.g., by compressing the handle in the variation shown in FIGS. 9A-9D), and the device can be withdrawn or re-positioned.

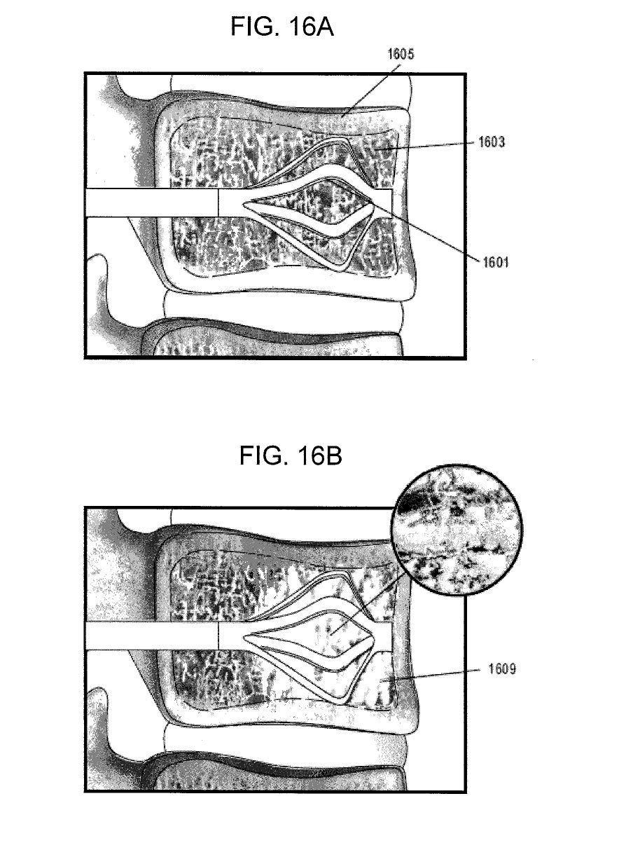

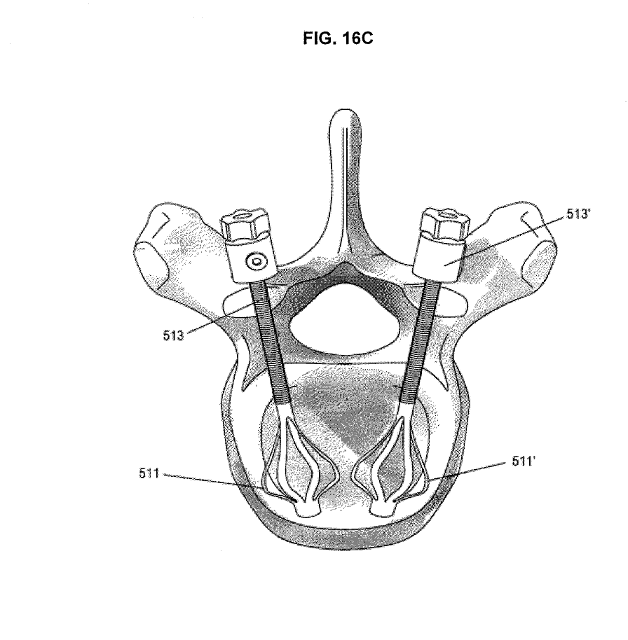

As mentioned above, a cement or additional supporting material may also be used to help secure the stabilization device in position and repair the bone. For example, bone cement may be used to cement a stabilization device in position. FIGS. 16A-16C illustrate one variation of this. In FIG. 16A the stabilization device 1601 has been expanded within the cancellous bone 1603 and is abutting the cortical bone 1605. Although in some variations the addition of the stabilization device may be sufficient to repair the bone, it may also be desirable to add a cement, or filler to help secure the repair. This may also help secure the device in position, and may help close the surgical site.

For example, in FIG. 16B a fluent bone cement 1609 has been added to the cancellous bone region around implant. This cement will flow through the channels of trebeculated (cancellous) bone, and secure the implant in position. This is shown in greater detail in the enlarged region. This bone cement or filler can be applied using the delivery cannula (e.g., through a cement cannula, as described above), and allowed to set.

While preferred embodiments of the present invention have been shown and described herein, such embodiments are provided by way of example only. Numerous variations, changes, and substitutions are possible without departing from the invention. Thus, alternatives to the embodiments of the invention described herein may be employed in practicing the invention. The exemplary claims that follow help further define the scope of the systems, devices and methods (and equivalents thereof).

The devices and methods for treating vertebral bodies describes above in detail may be used for the implantation of a self-reshaping device through a pedicle into the cancellous bone interior of a vertebral body, as mentioned. The self-reshaping of embodiments of the device includes a coincident longitudinally shortening of the device as a whole, and a radial expansion of struts. Following implantation and release from constraints that maintain the linear configuration, the struts of device self-expand, and while expanding, they cut through cancellous bone so as to arrive at the inner surface of the surrounding cortical bone of the superior (or cephalad) and inferior (or caudal) endplates of the vertebral body. The device may be sized and configured such that self-expansion takes the device to an appropriate dimension for the vertebral body. Thus, as the device approaches its final expanded dimension, it presses the surface outwardly so as to restore the height and volume of the vertebral body toward the dimensions of the vertebral body prior to the fracture.

FIG. 16C illustrates two stabilization devices 511, 511′ inserted bilaterally into a spinal segment. A pedicle (bone) screw 513, 513′ (attached through a pedicle of a vertebral body) has been attached into each stabilization device. Thus, in any of the variations described, the distal end of the device may also include a bone screw attachment region, so that a pedicle screw may be stabilized both at the proximal and the distal ends of the device. A bone screw may be inserted completely through the stabilization device, and may extend from the distal end. In some variations, the central region of the device includes a continuous (or mostly continuous) channel into which the bone screw may pass. [000138] In one variation of the method described herein, two self-expanding devices may be inserted bilaterally into a compression-fractured vertebral body for the purpose of restoring the height of the vertebra and expanding the body of the vertebra to restore it to its pre-fractured configuration. A compression fracture of a vertebral body typically reduces the height of a vertebral body; this compressed height will generally be referred to as H1. Upon implantation and expansion of a self-reshaping vertebral body stabilization device, the height of the vertebral body at the side or site of implantation is increased to a height H2. The height H2 is typically toward or an approximation of the height of the vertebral body prior to its state of compression.

Methods of using the implants, applicators and systems including them may include a step of selecting devices appropriate in form, shape, and size for each implantation site. Thus, in some variations the applicator or inserter devices described herein may be configured so that they may be used with implants of different sizes (both length and/or widths). For example, the devices may be configured so that the relative movement and separation of the inner and outer members spans a variety of sizes (e.g., lengths) of the bone stabilization implants from expanded to collapsed lengths. In some variations the handles include a limiter that prevents overexpansion of the applicator when coupled to an implant.

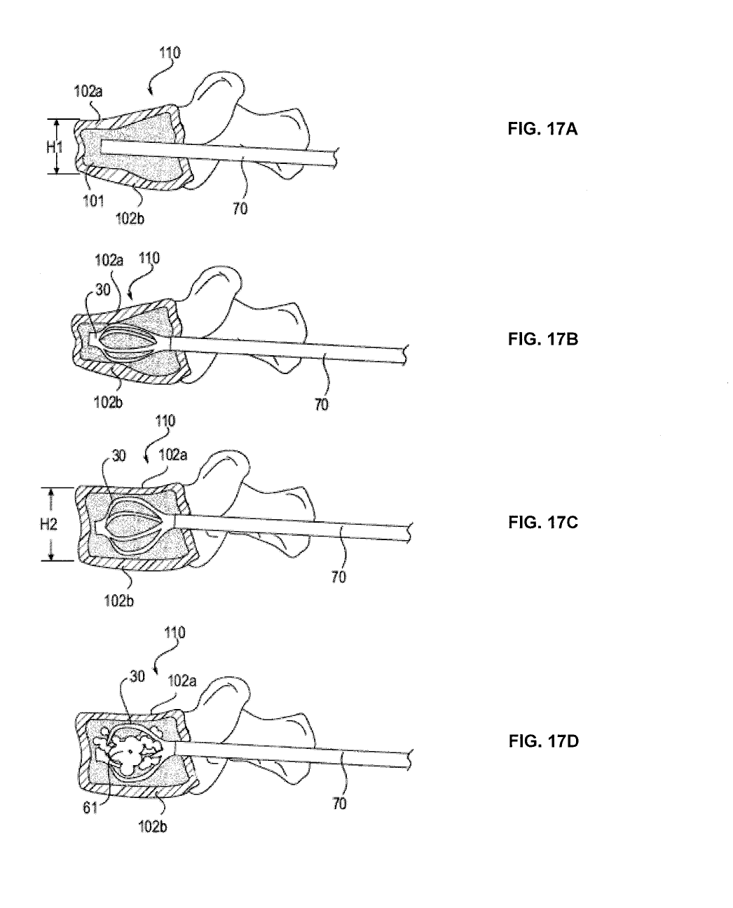

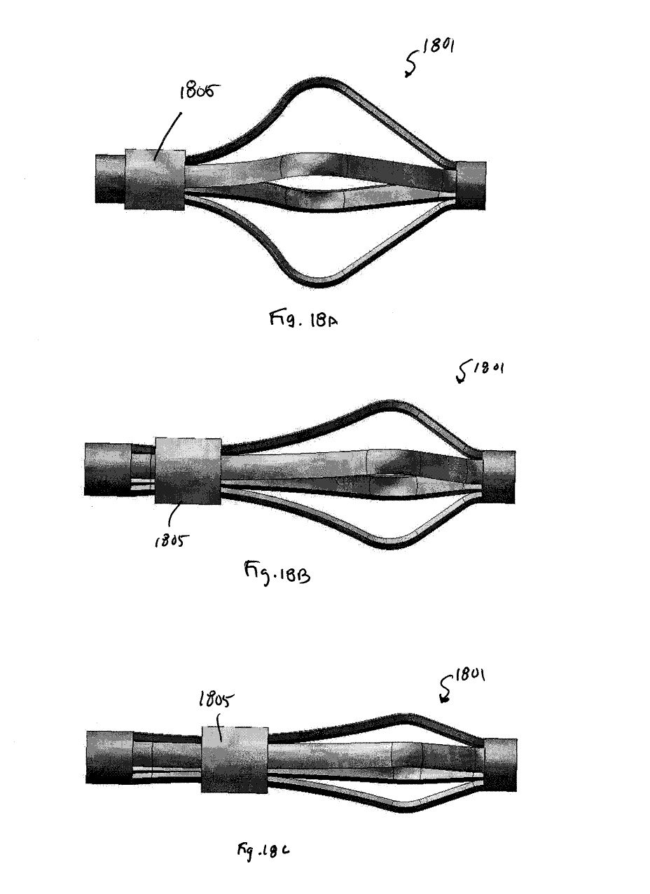

FIGS. 17A-17D show a series of lateral views of a vertebral body 110 with a height H1 (anterior on the left, posterior on the right) at a cross-section along a sagittal plane near a pedicle of the vertebral body. The vertebral body 110 has an outer layer of cortical bone, including a superior endplate 102a and an inferior endplate 102b, and an interior region including cancellous bone 101. FIG. 17A shows insertion of a deployment device 70 into a pre-drilled channel, a self-reshaping vertebral body stabilization device contained (not shown) within the deployment device. FIG. 17B shows an early point in the deployment of a self-reshaping vertebral stabilization device 30, with expandable struts beginning to expand. FIG. 17C shows full expansion of the expandable struts of the self-reshaping device 30 and consequent restoration of vertebral body to a height H2. FIG. 17D shows injection of a stabilizing material 61 into the space within the expanded struts of the self-reshaping device 30 and into available space within bone cancellous bone 101 surrounding the device. The material physically stabilizes the position of the device in the bone, stabilizes local bone that has been disrupted, and may also provide a matrix for the in-growth of bone, which further contributes to the stabilization of the device.

### Handles

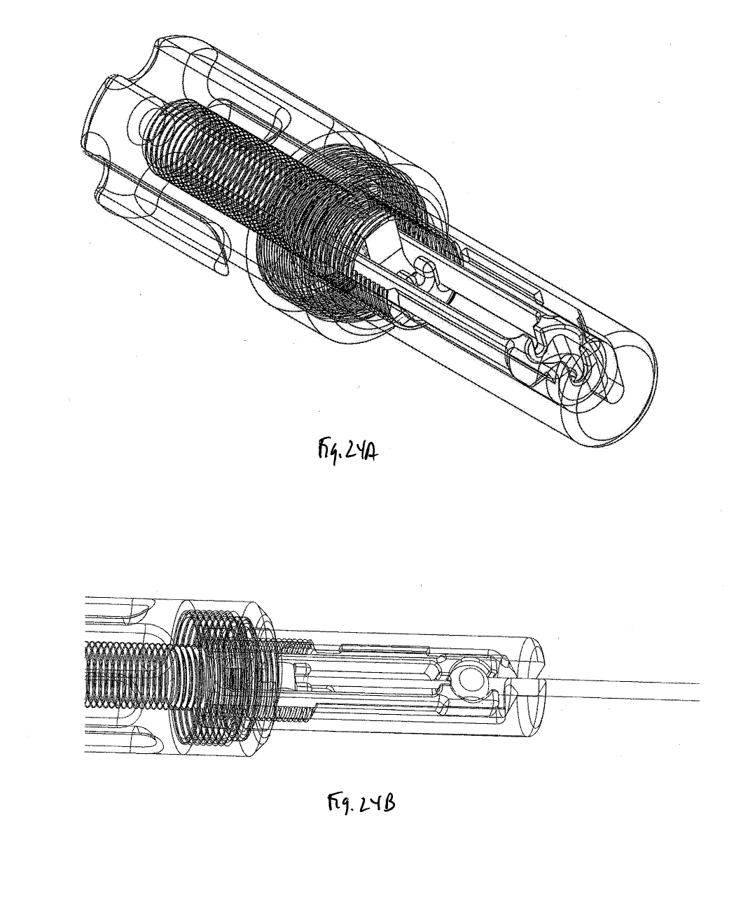

FIGS. 8A-8E and 9B and 9D illustrated one variation of a handle of an applicator, as described above. Other variations of handles, and particularly removable or reusable handles, are shown and describe in FIGS. 19A-30.

In general, these handles include a capture mechanism for connecting to the elongate member (e.g., inserter) that connects to the implant. As mentioned, an elongate member may be referred to as a delivery device or an elongate linkage member of the applicator. The elongate member typically includes a first elongate member (e.g., an outer member or cannula) that is configured to removably secure or couple with one region of an implant (e.g., the proximal end of the implant), and a second elongate member (e.g., an inner member, cannula or rod) that is configured to removably secure or couple with a second region of the implant (e.g., the distal end of the implant). The first and second members of the elongate member may be configured to couple and uncouple from the implant by rotating in opposite directions. The proximal end of the elongate member may include a proximal grips or couplers for grasping and/or manipulating the inner and outer members to control the expansion or contraction of the implant. The linkage portion of the applicator connects distally to the proximal and distal regions of the implant, and the handle engages the proximal end of the linkage portion of the applicator by connecting to and controlling these proximal couplers.

For example, the FIG. 19A shows a cross-section through one variation of a handle coupled to an elongate linkage portion 1901. The inner rod of the elongate linkage portion 1901 is connected to a ball-shaped grip 1905, while the outer cannula of the elongate linkage portion 1901 terminates proximally in a second grip region that is approximately rectangular 1907. In some variations (as described herein) one or both of these grip regions may be keyed so that the rotation of the inner and outer members can be controlled. In FIG. 19A, the relative longitudinal translation of the inner and outer elongate members of the linkage portion are controlled. For example, the handle includes a threaded rod or drive shaft 1921 that can be rotated by rotation of an adjustment knob 1923 at the proximal end. The threaded rod is a rotary gear that moves in rotation only, and does not translate along the longitudinal axis. The handle includes a latch 1933 for locking the expansion position of the implant by locking the internal moving slider 1935. This latch lock may be configured to prevent removal of the device from the handle when the implant is under tension by the handle (e.g., held collapsed). In some variations, the handle may have release to release the tension on the implant (e.g., held by the linkage portion of the applicator) before it may be released from the handle.

The diameter of the drive shaft, as well as the threads per inch, can be configured to control the mapping of the lateral movement and rotation of the adjustment knob based on implant size. For example, a typical implant may require a lateral change of approximately 1.4 mm to change from a collapsed (delivery) configuration to an expanded (deployed) configuration. The movement of the inner member relative to the outer member may be geared by adjustment of the dimensions so that an exact and convenient movement between the adjustment knob and the implant can be created.

FIG. 19B shows another variation of a handle similar to the variation shown in FIG. 19A. In this example, the handle includes the features described above, but is further configured so that it is compatible with only a single ‘size’ of implant, based on the separation between the proximal ends of the inner and outer members of the linkage portions of the applicator. A different length push rod 1921 is used for each size implant, adjusting the length of the seating components 1951, 1953 for the grip regions of the elongate linkage portion 1961. In some variations, the handle may be configured to work with a variety of different-sized implants. For example, in a variation such as the one shown in FIGS. 19A and 19B, the handle may be configured so that the push rod is adjustable.

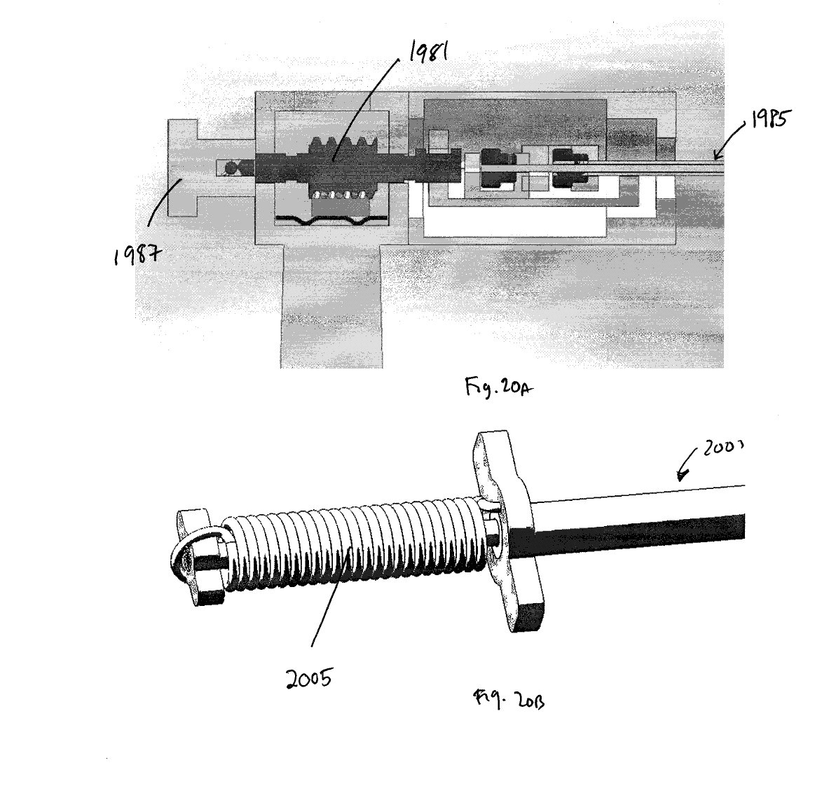

FIG. 20A shows another variation of a handle similar to those shown in FIGS. 19A and 19B. In FIG. 20A, some of the details are omitted for clarity. In this example, the drive shaft can be disengaged from driving the delivery shaft (elongate linkage portion) 1985 and push rod 1981. When the drive shaft is disengaged, the user can rotate the adjustment knob 1987 clockwise to detach one end of the implant from the delivery device and counterclockwise to detach the other end of the implant, in variations in which the implant proximal and distal coupling ends are counter-matched, as described above. In this variation, the drive-shaft engagement may be spring-loaded so that the default condition is that it is engaged. As mentioned above, the handle may include a safety lock to prevent disengaging the drive shaft when force is being applied to hold the implant in the delivery configuration.

In some variations the delivery device (also referred to herein as the elongate linkage portion 2001), including an outer member (e.g., cannula) and an inner member (e.g., rod or cannula), that couples to the implant distally and the handle proximally, may also include a bias 2005 that maintains a load on the implant when it is connected. For example, FIG. 20B illustrates one variation of an elongate linkage portion including a bias. In this variation, the proximal end of the elongate linkage portion includes a bias (e.g. spring) that tends to keep the distal ends of the outer elongate member and the inner elongate member separated (e.g., helping hold an attached implant in a pre-biased delivery configuration by pulling the proximal end of the elongate linkage portion together. Biasing the elongate linkage portion in this manner may be helpful to decrease the force needed to be provided by a user to hold the implant in the delivery configuration.

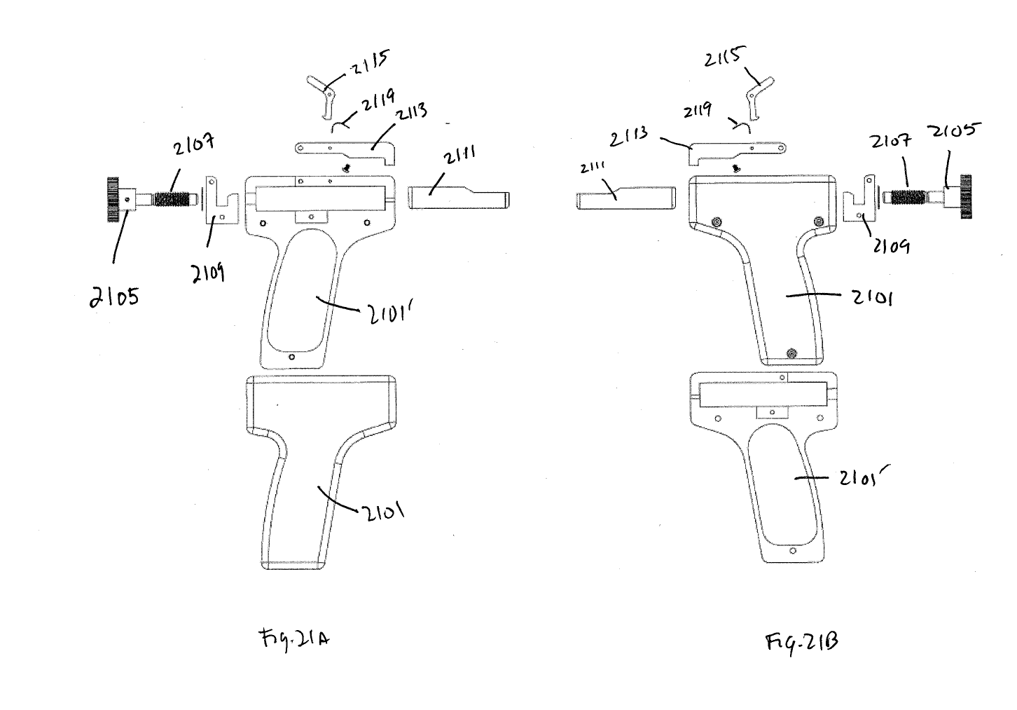

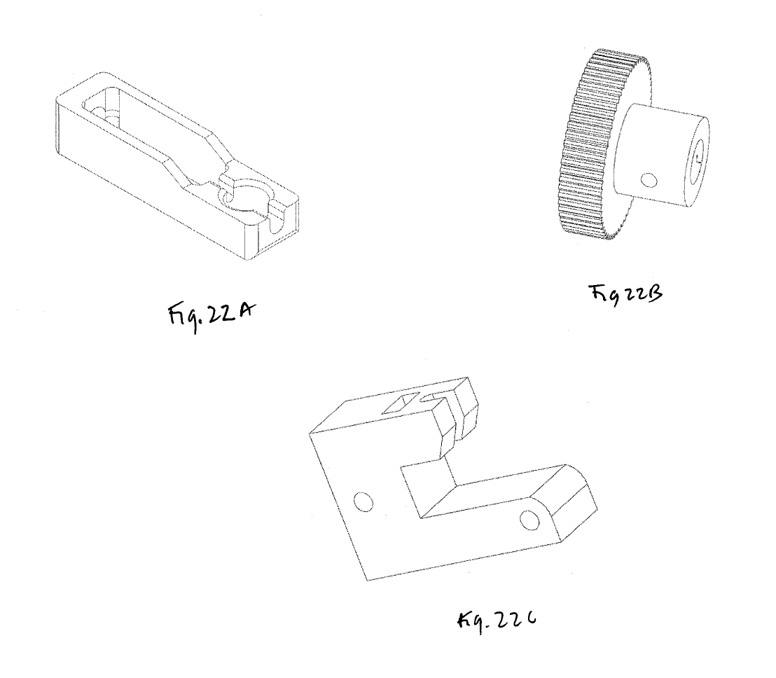

FIGS. 21A and 21B illustrate front and back exploded views of one variation of a handle similar to the variation shown in FIGS. 19A-20A. For example, in FIG. 21A, the handle includes a grip region 2101, 2101′, an adjustment knob 2105 (shown enlarged in FIG. 22B), a drive shaft 2107 coupled to the adjustment knob 2105, a sliding receiver 2109 for the inner member of the elongate linkage portion, and a fixed receiver 2111 for the outer member of the elongate linkage portion, and a latch 2113 for locking the relative positions of the inner and outer members, as well as a lock 2115 for the latch. The lock mechanism also includes a spring element 2119 for biasing the latch closed or opened until it is engaged. In the variation shown in FIGS. 21A and 21B, the handle is configured so that the sliding receiver 2109 seats the grip (which may be keyed or unkeyed) of the inner member (e.g., rod) of the delivery device/elongate linkage portion (not shown), and the fixed receiver 2111 seats the grip (which may be keyed or unkeyed) of the outer member (e.g., cannula) of the delivery device/elongate linkage portion. This configuration may be reversed. For example, the sliding receiver may be configured to seat the grip of the inner member (e.g., rod) of the elongate linkage portion and the fixed receiver may be configured to seat the grip of the outer member (e.g., cannula). FIG. 22A shows an enlarged perspective view of the fixed receiver and FIG. 22C shows an enlarged perspective view of the movable receiver.

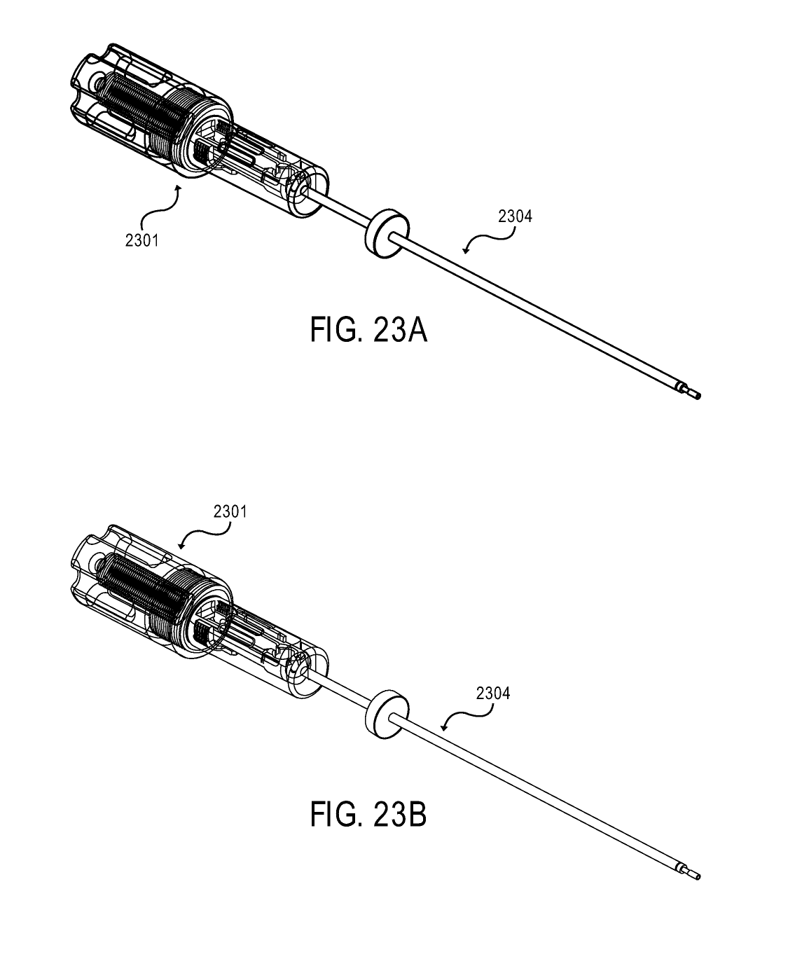

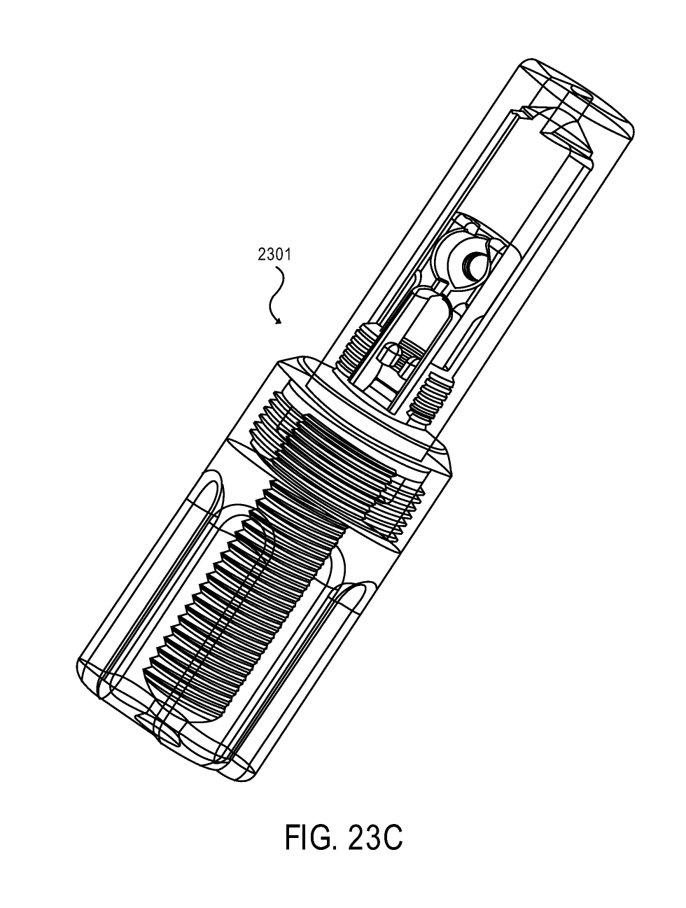

FIGS. 23A-23C illustrate another variation of a handle. The handle 2301 shown in FIG. 23C is configured to couple with the proximal end of an elongate linkage portion of an applicator. A handle such as the one shown in FIG. 23C includes one or more sections that are rotatable relative to other regions of the handle (or a relative to the coupled elongate linkage member). Rotation of a portion of the handle may controllably move the inner member of the elongate linkage member relative to the outer member of the elongate linkage member (or vice versa), allowing the implant to be controllably self-expanded (e.g., deployed) or alternatively collapsed (e.g., for removal). FIGS. 23A and 23B illustrate the handle 2301 coupled to an elongate linkage member 2304.

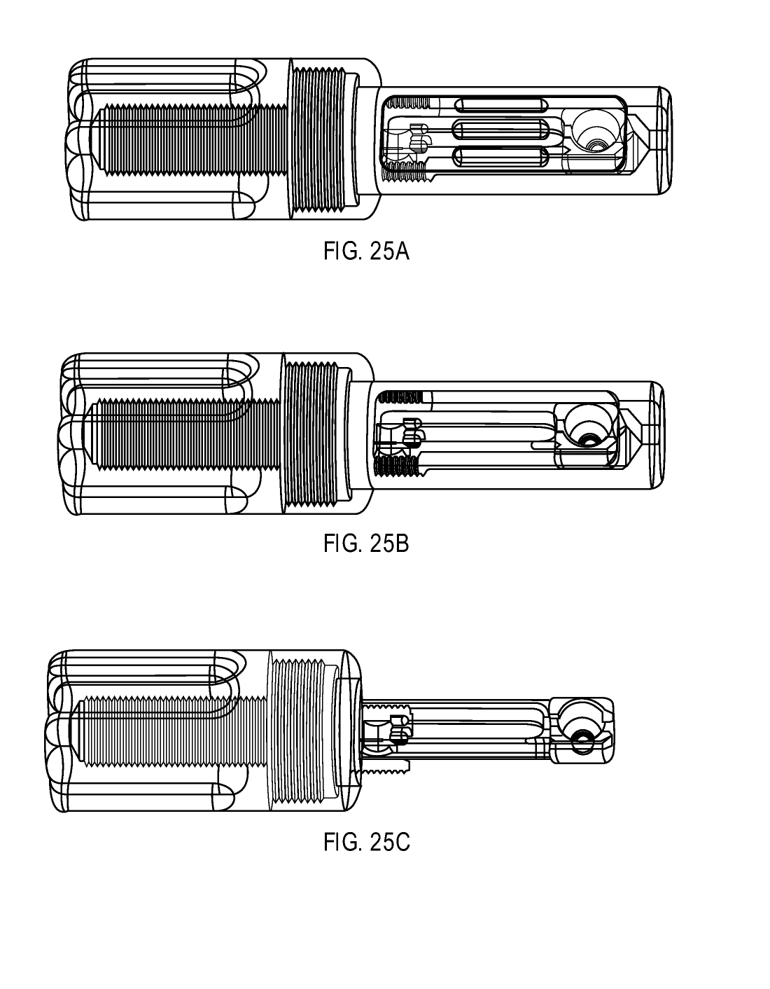

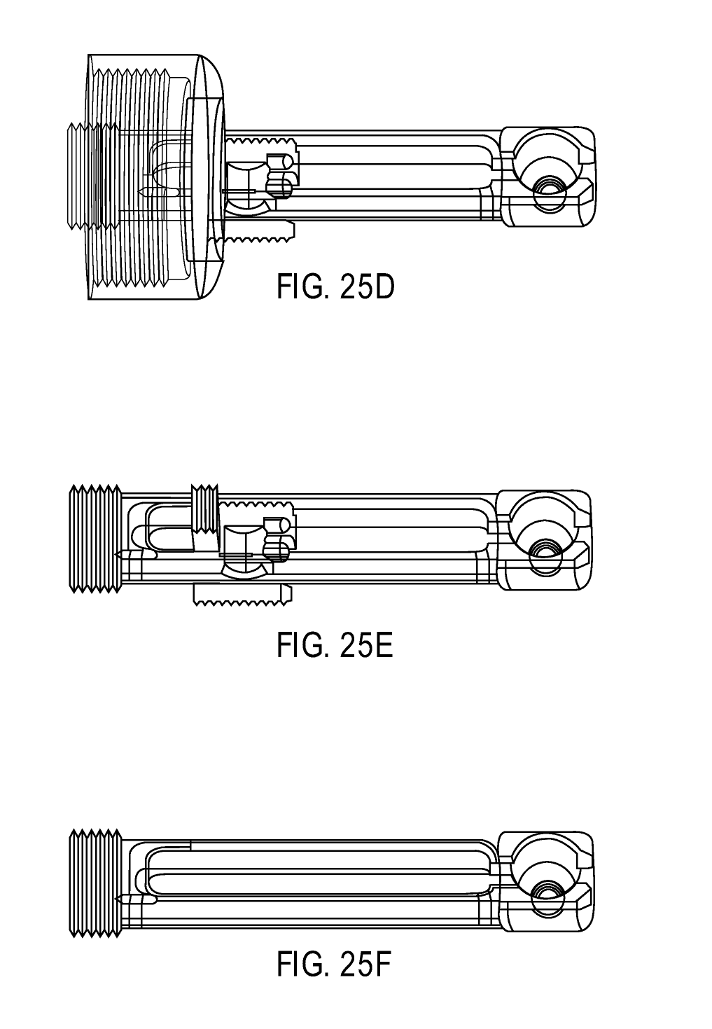

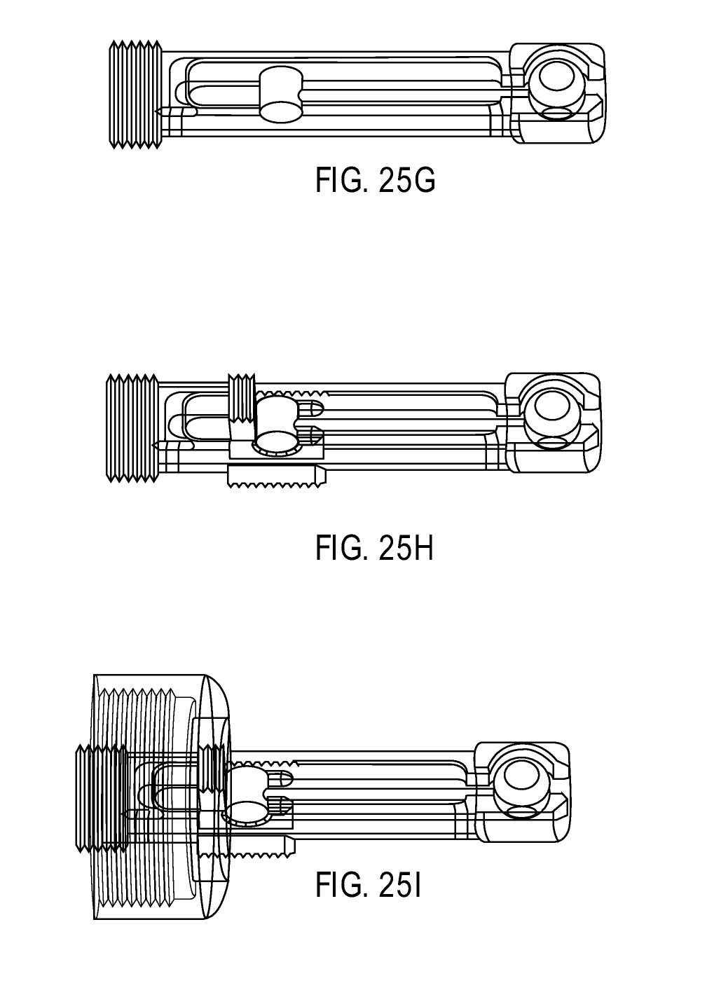

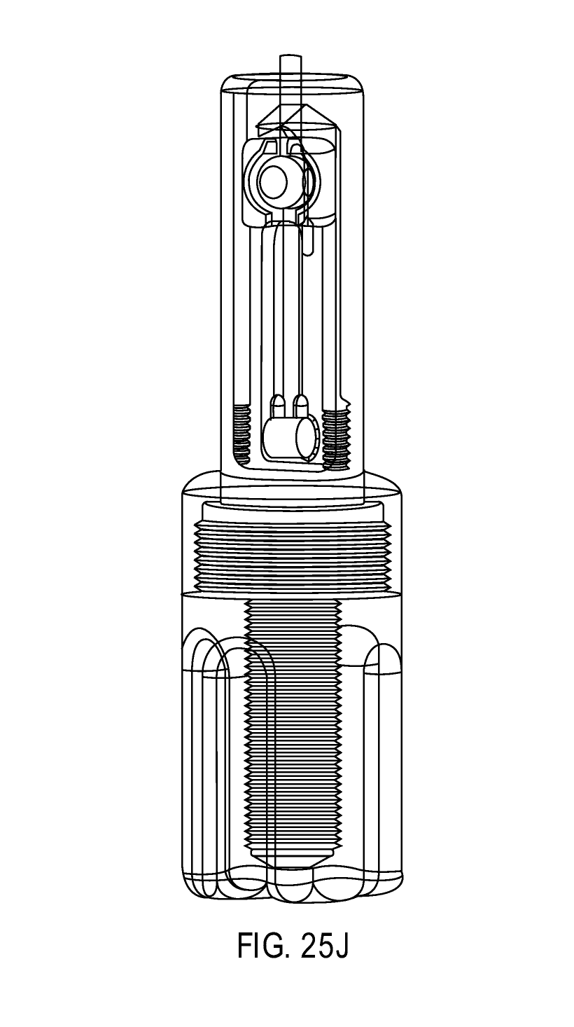

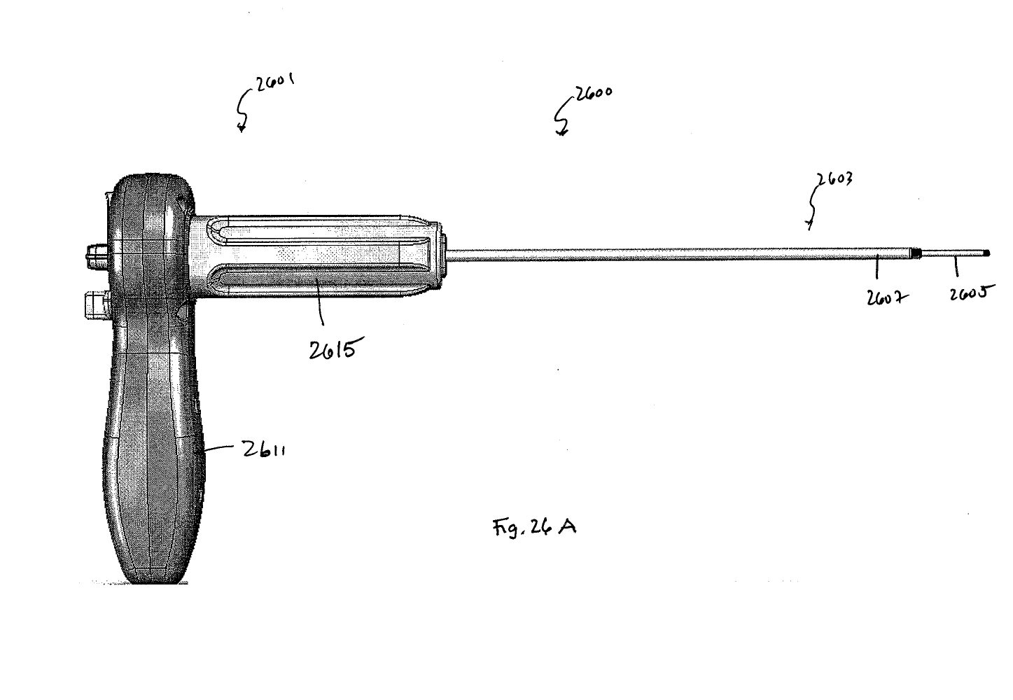

The exemplary handle 2101 shown in FIG. 23A-23C includes only six components, though more or fewer components may be used. FIGS. 25A-25K illustrate the relationship between each of these components (and the inserter distal end). As mentioned above, the handles may be configured to be reusable/durable, or they may be configured as single-use.

In some variations the handle is permanently affixed to the elongate linkage member (e.g., forming a unitary applicator); in other variations the elongate linkage member of the inserter is separate from the handle.

In use, a handle that is detachably coupleable to an elongate linkage member may be attached within the handle, e.g., by removing a handle cover (see FIG. 25B). The cover may be replaced to secure the proximal ends of the inserter in place. Once the inserter is secured in position, the handle (E.g., the proximal end) may be rotated to allow the proximal end of the implant to controllably move towards the distal end, allowing the implant to expand. Rotation in the opposite direction moves the proximal end of the implant away from the distal end. A rotary gear may be include within the housing and configured to advance the first elongate member of the elongate linkage member.

In some variations, the handle may be configured as a ratcheting handle. A ratcheting handle may include a lever arm can engage the rotatable region of the handle and allow it to be rotated. The lever arm may provide a further mechanical advantage for collapsing or expanding a stabilization device. In some variations (not pictured), a portion of the handle may be removable so that he handle can be ratcheted from different angles or directions. In some variations, the handle may include a directional control for the ratchet mechanism, such as a button, lever, etc. Changing the setting on the directional control may allow the direction rotation to be changed, while the applied direction of rotation (e.g., pushing or pulling the level arm) is the same.

In some variations, the distal end of the stabilization device is connected to an inner member of the inserter. For example, the inner member of the inserter may be a rod that is relatively fixed as an outer rod or cannula may be moved around it (or along it). Thus, the shaft (e.g., the hollow outer part) moves to expand/contract the stabilization device.

In addition to the inserters (e.g., handles and elongate linkage members) described and illustrated above, other variations of inserters may also be used. An inserter may include a threaded outer member that is configured to secure to the proximal end of the stabilization device. In this example, a handle may be configured to mate with the threaded outer portion of the inserter, For example, this may eliminate the threading in the handle. This threading may be keyed to prevent rotation of the inserter. Preventing rotation, particularly unnecessary rotation, may prevent the device from unthreading prematurely at the distal end. In some variations the keying may be a channel, etc.

In any of the variations described herein, the handles (or other portions of the inserter) may be marked or coded to indicate the size of the implant. For example, the handle (which may mate with a generic handle, regardless of the size of the attached stabilization device) may be marked with numbering/lettering to indicate the size, and/or color coated. In some variations the handle is marked to indicate the orientation of the implant (e.g., the self-expanding struts) relative to the inserter.

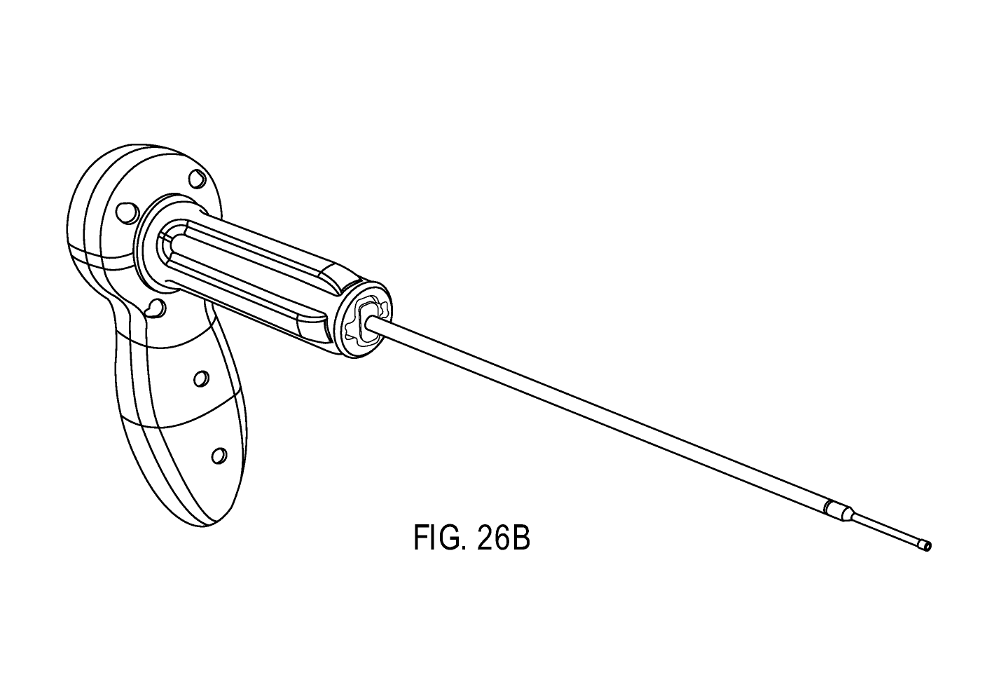

FIGS. 26A-30 illustrate another variation of an inserter for inserting a bone support implant. This variation is similar to the device shown in FIG. 1. In this variation, the inserter 2600 includes a handle region 2601 and an elongate linkage member 2603. The elongate linkage member include an inner member 2605 and an outer member 2607. The distal ends of the inner and outer members are threaded to couple to end regions (proximal and distal) of an implant as described above.

The handle 2601 shown in FIG. 26A is a ratcheting handle configured to connect to the elongate linkage member 2603. FIG. 26B shows a side perspective view of the inserter shown in FIG. 26A. This handle includes a perpendicular handle region 2611 and a ratchet grip region 2615.

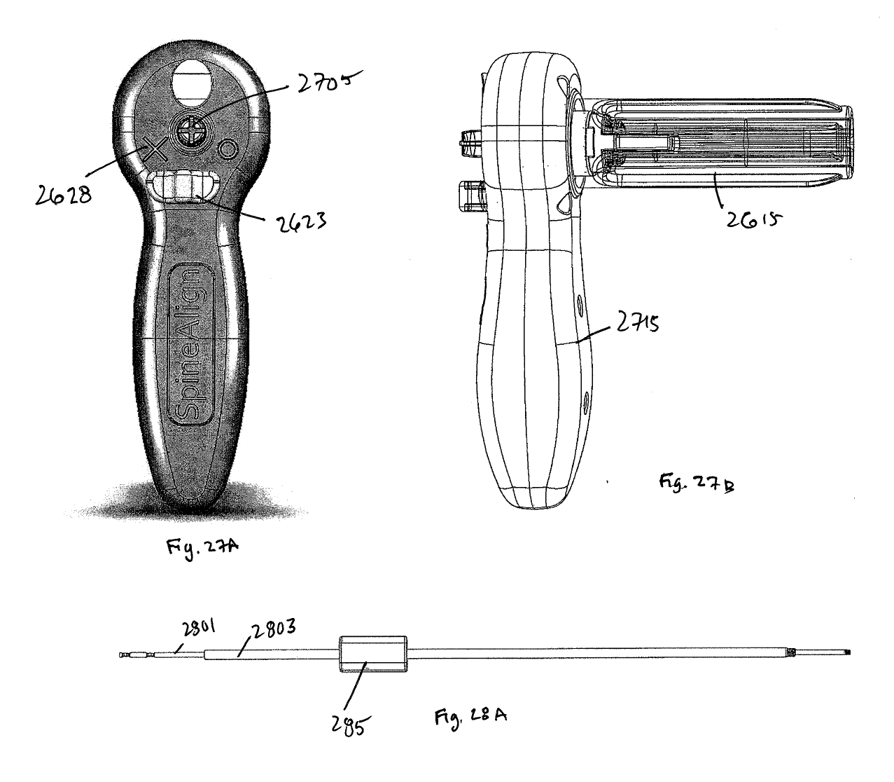

FIG. 27A illustrates a back view of the handle shown in FIGS. 26A-26B. The operation of this device will be described in greater detail below. The device includes a ratchet switch 2623 that changes the direction of the ratcheting mechanism so that the handle turns to engage the implant in expansion or collapse (e.g., driving the distal ends of the inner and outer member of the elongate linkage member either apart or towards each other). The handle shown in this example also includes an indicator of the orientation 2628 of the struts on the implant. Thus, the implant may be loaded onto the elongate linkage member, and therefore the handle, in a manner that maintains the orientation of the implant.

The handle shown in FIG. 27A also includes a release control or mechanism 2705 that operates as an “escape hatch” safety feature. In this example, the release mechanism may be unscrewed from the handle to release the elongate linkage member from the handle in the event that the handle fails (e.g., jams, locks, or the like). Activation of the release mechanism releases any force applied by the handle. Thus, the implant (connected to the elongate linkage member) may be removed from the handle.

In some variations the handle may also include an indicator of the size of the implant to be used (e.g., 10 mm, 12 mm, 16 mm, 18 mm, etc.). In some variations the system includes one or more sensors or connections to sensors. For example, the handle may include a connector to a temperature sensor or other sensor (including visualization devices) for sensing data from the implant or the region of implantation.

FIG. 27B illustrates a side perspective view of the handle shown in FIG. 27A. The ratchet handle 2615 is shown as partially transparent. In use, the ratchet handle may be rotated relative to the handle body 2715 to advance or withdraw the inner and outer members of the elongate linkage member, and thereby expand/contract the implant. The ratchet mechanism internal to the handle includes a limiter to prevent it from being overextended in either direction, protecting the device from over-expansion or over-collapsing, which may lead to breaking of the implant. In some configurations the handle may be preset for use with a particular size implant. In other variations, the handle may be configured to be switched to selected sizes.

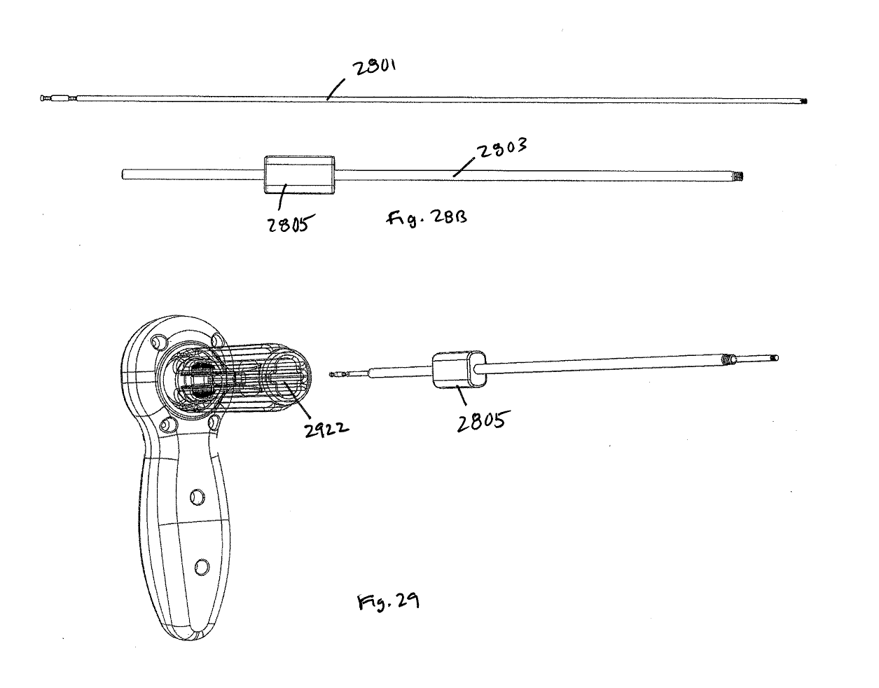

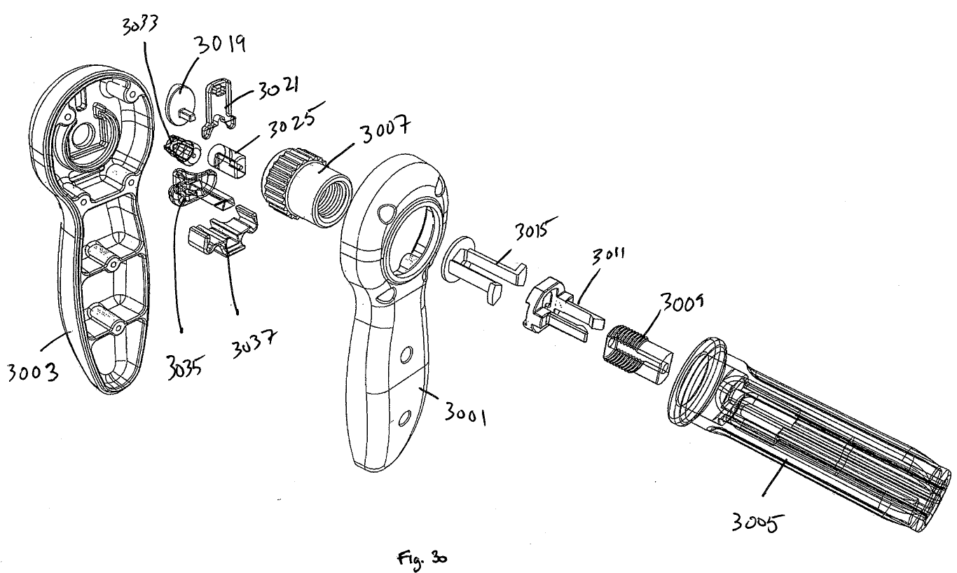

FIGS. 28A and 28B show one variation of an elongate linkage member portion of an applicator. In FIG. 28A, the elongate linkage member includes an inner member 2801 and an outer member 2803. The outer member includes a keyed engagement member 2805, which may be referred to as a shaft stabilizer. The keyed engagement member is configured to mate with the handle (as illustrated in FIG. 29) and maintain the orientation of the implant at the distal end of the applicator. It may also stabilize the shaft of the elongate linkage member (e.g., the outer member) within the handle. Thus, the handle may include a mating region 2922 at the distal end (e.g., opening into the handle) configured to mate with the shaft stabilizer 2805.

FIG. 30 shows an exploded view of the handle portion of the applicator shown in FIGS. 26A-29. In FIG. 30, the handle includes: a front and back handle grip region 3001, 3003; a ratchet grip region 3005; a shaft driver overmold element 3009; a retainer 3011; a retainer attachment 3015; the ratchet mechanism 3007; a release switch 3019; a rod release 3021; a rod (inner member) stop 3025; a rod end cap 3033; a ratchet direction switch 3035 and a ratchet direction pawl 3037.

In operation, the applicator may be connected to the proximal and distal ends of an implant by connecting to the elongate linkage member, as mentioned above. The proximal end of the implant may connect to the outer member, while the distal end of the implant connects to the inner member (e.g., rod). Both ends may include counter-directional threads. The threads may be on the outer surface of the proximal end and on the inner surface of the distal end. The implant may be connected to the elongate linkage member either before or after it has been coupled to the handle. In some variations the elongate linkage member is pre-packaged coupled to the implant, so that it may be opened from a sterile packaging for use. The same handle may be re-used for different implants, typically within the same patient.

A self-expanding implant, connected to the applicator as described above, may be inserted into a patient by manipulating the handle and shaft of the applicator. Once it is positioned as desired (which may be visualized by florosocopy), it may be allowed to controllably self-expand using the applicator. As mentioned, the applicator may include an indicator of the orientation of the self-expanding struts. Thus, the handle and shaft of the applicator may be manipulated (e.g., rotated) orient the implant so that the struts will be positioned as desired.

The elongate linkage member may be connected to the handle by engaging the keyed engagement member (shaft stabilizer) on the surface of the elongate linkage member. Inserting the shaft stabilizer into the handle also engages the inner and outer members of the elongate linkage member. Thereafter, rotation of the ratcheting handle will move the outer member, and therefore the proximate end of the implant, relative to the inner member. The direction of motion may depend on the ratchet switch, which moves the pawl member to select the engaged motion of the ratchet mechanism.

In some variations it is helpful that the proximal end of the implant is moving relative to the length of the implant. By moving the proximal end, the implant may be inserted into a desired location and controllable allowed to self-expand into a position without extending from the distal implantation location. Thus, the implant will not shift position relative to the distal insertion site by foreshortening as the implant is controllably self-expanded into a deployed configuration.

The ratchet direction may be selected and switched using the ratcheting switch as indicated. In some variations, an indicator (e.g., a symbol, color, text, etc.) may indicate the direction of movement enabled (e.g., expansion/deployment or contraction/retraction of the implant).

Once the implant has been inserted and allowed to self-expand, the applicator (handle and shat of the elongate linkage member) may be removed. The force applied to the implant by the handle may be released by pushing the release button (switch), on the handle, so that the shaft of the elongate linkage member may be removed from the handle. The handle may be removed from the elongate linkage member and the elongate linkage member may then be removed from the implant by the proximal and distal ends. In some variations the implant may be removed from the elongate linkage member while still attached to the handle. In other variations the handle is removed first. The elongate linkage member may be decoupled from the proximal and distal ends of the implant by rotating the inner and outer members (e.g., counter clockwise at the distal end and clockwise at the proximal end) in threaded variations.

If the position of the implant is not optimal, the position may be re-adjusted using the handle as indicated above, e.g., by collapsing the implant using the handle and moving the implant.

The methods, devices and systems described herein provide only some variations described herein, and additional variations may be included and are contemplated. While embodiments of the present invention have been shown and described herein, such embodiments are provided by way of example only. Thus, alternatives to the embodiments of the invention described herein may be employed in practicing the invention. The exemplary claims that follow help further define the scope of the systems, devices and methods (and equivalents thereof).

What is claimed is:

1. A rotary applicator handle for delivery or removal of a bone stabilizing implant that is distally coupled to an elongate linkage member, the handle comprising:
a handle grip configured to be held in the palm of a hand;
a housing at least partially surrounding a first seat configured to hold the proximal end of a first elongate member of the elongate linkage member and a second seat configured to hold the proximal end of a second elongate member of the elongate linkage member;
a rotary gear within the housing, the rotary gear configured to drive the axial motion of the first member of the elongate linkage member relative to the second member of the elongate linkage member; and
a rotatable control coupled to rotary gear and configured to rotate the rotary gear.

2. The rotary applicator handle of claim 1, wherein the rotary gear is a ratcheting gear comprising a pawl.

3. The rotary applicator handle of claim 1, further comprising a directional switch coupled to the rotary gear and configured to control direction of axial motion driven by the rotary gear.

4. The rotary applicator handle of claim 1, wherein the rotary gear comprises a drive shaft.

5. The rotary applicator handle of claim 1 further comprising an indicator to indicate the orientation of the bone stabilizing implant relative to the handle.

6. The rotary applicator handle of claim 1 further comprising a release control configured to release the elongate linkage member from the handle.

7. The rotary applicator handle of claim 1 further comprising a force release control configured to release the axial force applied to the elongate linkage member by the handle.

8. The rotary applicator handle of claim 1 further comprising a mating region configured to mate with a shaft stabilizer on the first member of the elongate linkage member.

9. The rotary applicator handle of claim 1, wherein the rotatable control comprises a rotatable control grip.

10. The rotary applicator handle of claim 1, wherein the rotary gear is configured to axially move the second seat relative to the first seat so that the proximal end of an implant coupled to the first member of the elongate linkage member moves while the distal end of the implant remains relatively stationary.

11. A ratcheting applicator handle for delivery or removal of a bone stabilizing implant that is distally coupled to an elongate linkage member, the handle comprising:
a first handle grip region;
a housing at least partially surrounding a first seat configured to hold the proximal end of an inner member of the elongate linkage member and a second seat configured to hold the proximal end of an outer member of the elongate linkage member;
a ratcheting gear within the housing, the ratcheting gear configured to drive the axial motion of the outer member of the elongate linkage member relative to the inner member of the elongate linkage member;
a rotatable grip coupled to ratcheting gear and configured to rotate the ratcheting gear; and
a directional switch coupled to a pawl and configured to select the axial direction that the outer member is driven relative to the inner member.

12. An inserter system for delivery or removal of a bone stabilizing implant, the inserter comprising:
an elongate linkage member configured to distally couple with the bone stabilizing implant, the elongate linkage member comprising:
a first elongate member configured to releasably couple at its distal end with the proximal end region of the bone stabilizing implant; and
a second elongate member configured to releasably couple at its distal end with the distal end region of the bone stabilizing implant; and
a rotary handle, the handle comprising:
a handle grip region;
a housing at least partially surrounding a first seat configured to hold the proximal end of the first elongate member and a second seat configured to hold the proximal end of the second elongate member;
a rotary gear within the housing, the rotary gear configured to drive the axial motion of the first member relative to the second elongate member; and
a rotatable control configured so that rotation of the rotatable control moves the rotary gear.

13. The inserter system of claim 12, wherein the first member comprises an outer cannula and the second elongate member comprises an internal rod.

14. The inserter system of claim 12, wherein the elongate linkage member further comprises an end grip at the proximal end of the first elongate member that is keyed to fit within the first seat of the rotary handle.

15. The inserter system of claim 12, wherein the rotary gear is a ratcheting gear comprising a pawl.

16. The insert system of claim 12, further comprising a directional switch coupled to the rotary gear and configured to control the direction of axial motion driven by the rotary gear.

17. The system of claim 12, further comprising a self-expanding implant having a plurality of self-expanding struts and a proximal attachment region configured to releasably attach to the first elongate member and a distal attachment region configured to releasably attach to the second elongate member.

18. A method of collapsing and expanding a self-expanding implant, the method comprising:
seating the proximal end of an elongate linkage member within a rotary applicator handle so that the proximal end of a first elongate member of the elongate linkage member is held within a first seat and the proximal end of a second elongate member of the elongate linkage member is held within a second seat; and
rotating a control on the rotary applicator handle to drive a rotary gear that axially moves the first elongate member relative to the second elongate member so that the proximal end of a self-expanding implant that is coupled to the distal end of the first elongate member is moved relative to the distal end of the self-expanding implant that is coupled to the distal end of the second elongate member.

19. The method of claim 18, wherein the step of rotating the control on the rotary applicator handle comprises limiting the axial motion of the first elongate member relative to the second elongate member to prevent damage to the self-expanding implant.

20. The method of claim 18, wherein the step of rotating the control on the rotary applicator comprises moving the first elongate member relative to the second elongate member without substantially moving the second elongate member.

21. The method of claim 18, wherein the step of rotating the control on the rotary applicator handle comprises driving a ratcheting rotary gear comprising a pawl.

22. The method of claim 18, further comprising selecting the direction of axial motion by switching a ratchet switch that is coupled to a pawl.

23. The method of claim 18, further comprising activating a control on the rotary applicator handle to release the axial force applied to the elongate linkage member by the rotary applicator handle.
